(19) United States
(12) Patent Application Publication
(10) Pub. No.: US 2013/0122045 A1
(43) Pub. Date: May 16, 2013

(54) Cross-Protective Influenza Vaccine

(71) Applicants: Tim Hirst, Yarralumla (AU); Mohammed Alsharifi, Gilles Plains (AU); Arno Mullbacher, Curtin (AU)

(72) Inventors: Tim Hirst, Yarralumla (AU); Mohammed Alsharifi, Gilles Plains (AU); Arno Mullbacher, Curtin (AU)

(73) Assignee: Gamma Vaccines Pty Limited, Forrest (AU)

(21) Appl. No.: 13/643,995

(22) PCT Filed: Apr. 28, 2010
(86) PCT No.: PCT/AU10/00486

§ 371 Date: Jan. 2, 2013

(51) Int. Cl. A61K 39/145

(52) U.S. Cl. CPC A61K 39/145
USPC 424/209.1

(57) ABSTRACT

The invention relates to a method for preventing or treating an influenza virus infection in a subject, the method comprising administering to the subject a therapeutically effective amount of a gamma-irradiated influenza virus, wherein the virus comprises an A/Port Chalmers/1/73 (A/PC) strain backbone of PB2, PB1, PA, NP, M1, M2 and NEP proteins.

### TECHNICAL FIELD

The invention relates generally to the field of immunology and vaccines. More specifically, the invention relates to influenza vaccines and their use for preventing and treating influenza virus infection.

### BACKGROUND

Influenza is a highly contagious disease arising from infection of the respiratory tract by the influenza virus. Millions of people suffer from influenza infection in any given calendar year and the virus has been responsible for numerous epidemics and pandemics causing widespread morbidity and mortality.

Commercially-produced seasonal influenza vaccines are generally chemically-inactivated whole virus or subunit preparations designed to induce neutralising antibody responses against viral hemagglutinin (HA) and neuraminidase (NA) surface glycoproteins. However, these surface antigens are subject to frequent antigenic variation and differ significantly between influenza strains. Consequently, seasonal influenza vaccines are effective only against the specific influenza strains on which they are based thus offering limited protection and necessitating an accurate prediction of the predominant strains that will circulate in a forthcoming flu season. Furthermore, selective pressures imposed by seasonal flu vaccines favour the emergence of new strains with altered HA and NA proteins rendering the vaccines ineffective over a relatively short time period. An additional shortcoming of seasonal flu vaccines is an inability to provide immunity against newly arising strains that are often responsible for serious influenza epidemics and pandemics.

The failure of seasonal influenza vaccines to provide significant cross-protective immunity against multiple influenza subtypes arises at least in part from their inability to induce significant levels of T-lymphocyte-mediated immunity. T-lymphocyte responses are central to successful recovery from primary influenza virus infection and reduce the severity of symptoms by restricting viral load during the acute phase. More significantly, T-lymphocyte responses are predominantly directed against internal viral proteins which are highly conserved among all influenza virus subtypes and less susceptible to mutation. Hence, vaccines capable of inducing strong T-lymphocyte responses favour the development of long-lasting cross-protective immunity against both existing strains and new strains that may emerge during a given flu season. Apart from reducing the incidence of influenza infection, cross-protective influenza vaccines can be stockpiled to allow a swift response if potential influenza epidemics/pandemics begin to emerge. Accordingly, the ability to provide cross-protective immunity by inducing T-lymphocyte-mediated antiviral responses is a highly desirable characteristic that is not met by commercially available influenza vaccines.

A need exists for improved methods and vaccines for preventing and treating influenza which are capable of inducing broad cross-protective immunity against multiple influenza subtypes. Preferably, the improved methods and vaccines induce stronger cross-protective T-lymphocyte responses against influenza viruses than existing influenza treatment(s).

### SUMMARY OF THE INVENTION

Vaccines and treatments capable of inducing cross-protective immunity against multiple influenza subtypes and strains are highly desirable. The present inventors have determined that strong T-lymphocyte responses against conserved influenza virus proteins can be induced by administering influenza virus preparations inactivated by gamma-irradiation. Accordingly, the administration of gamma-irradiated virus preparations provides a means of inducing cross-protective immunity against multiple influenza subtypes and strains. Surprisingly, the present inventors have also determined that immunisation with a particular gamma-irradiated subtype of influenza virus (A/Port Chalmers/1/1973 (H3N2), also referred to hereinafter as “A/PC”) induces stronger cross-protective T-lymphocyte responses than immunisation with other gamma-irradiated influenza virus subtypes. It is postulated that the backbone of gamma-irradiated A/PC (made up of the internal viral proteins) may be responsible for inducing a significant proportion of the cross-protective T-lymphocyte responses observed.

Accordingly, in a first aspect the invention provides a method for preventing or treating an influenza virus infection in a subject, the method comprising administering to the subject a therapeutically effective amount of a gamma-irradiated influenza virus, wherein the virus comprises an A/Port Chalmers/1/73 (A/PC) strain backbone of PB2, PB1, PA, NP, M1, M2 and NEP proteins.

In a second aspect, the invention provides a method for inducing or enhancing cross-protective immunity against multiple influenza virus subtypes in a subject, the method comprising administering to the subject a therapeutically effective amount of a gamma-irradiated influenza virus, wherein the virus comprises an A/Port Chalmers/1/73 (A/PC) strain backbone of PB2, PB1, PA, NP, M1, M2 and NEP proteins.

In one embodiment of the previous aspects, the gamma-irradiated influenza virus is administered intranasally to the subject.

In another embodiment of the previous aspects, the gamma-irradiated influenza virus is administered in a freeze-dried form.

In a third aspect, the invention provides a gamma-irradiated influenza virus comprising an A/Port Chalmers/1/73 (A/PC) strain backbone of PB2, PB1, PA, NP, M1, M2 and NEP proteins, for preventing or treating influenza infection in a subject.

In a fourth aspect, the invention provides a gamma-irradiated influenza virus comprising an A/Port Chalmers/1/73 (A/PC) strain backbone of PB2, PB1, PA, NP, M1, M2 and NEP proteins, for inducing or enhancing cross-protective immunity against multiple influenza virus subtypes in a subject.

In a fifth aspect, the invention provides use of a gamma-irradiated influenza virus comprising an A/Port Chalmers/1/73 (A/PC) strain backbone of PB2, PB1, PA, NP, M1, M2 and NEP proteins, for the preparation of a medicament for preventing or treating influenza infection in a subject.

In one embodiment of the fourth and fifth aspects, the gamma-irradiated influenza virus is formulated for intranasal administration.

In one embodiment of the fourth and fifth aspects, the gamma-irradiated influenza virus is formulated in a freeze-dried form.

In a sixth aspect, the invention provides use of a gamma-irradiated influenza virus comprising an A/Port Chalmers/1/73 (A/PC) strain backbone of PB2, PB1, PA, NP, M1, M2 and NEP proteins, for the preparation of a medicament for inducing or enhancing cross-protective immunity against multiple influenza virus subtypes in a subject.

In one embodiment of the fifth and sixth aspects, the medicament is formulated for intranasal administration.

In on embodiment of the fifth and sixth aspects, the medicament is formulated in a freeze-dried form.

In one embodiment of the second, fourth, and sixth aspects, the cross-protective immunity comprises cross-protective cellular immunity.

In one embodiment of the second, fourth, and sixth aspects, the cross-protective cellular immunity comprises either or both of:

(i) a cross-protective helper T-lymphocyte response

(ii) a cross-protective cytotoxic T-lymphocyte response.

In one embodiment of the second, fourth, and sixth aspects, the cross-protective immunity comprises cross-protective humoral immunity.

In a seventh aspect, the invention provides a method of producing a cross-protective influenza vaccine, the method comprising inactivating a preparation of influenza virus by gamma-irradiation, wherein the virus comprises an A/Port Chalmers/1/73 (A/PC) strain backbone of PB2, PB1, PA, NP, M1, M2 and NEP proteins.

In one embodiment of the seventh aspect, the method comprises the additional step of freeze-drying the virus after inactivating by gamma-irradiation.

In an eighth aspect, the invention provides a cross-protective influenza vaccine comprising a gamma-irradiated influenza virus, wherein the virus comprises an A/Port Chalmers/1/73 (A/PC) strain backbone of PB2, PBI, PA, NP, M1, M2 and NEP proteins.

In one embodiment of the eighth aspect, the vaccine is in freeze-dried form.

In one embodiment of the seventh and eighth aspects, the vaccine is formulated for intranasal administration.

In one embodiment of the previous aspects, the influenza virus is purified by tangential/cross-flow filtration prior to gamma-irradiation.

In another embodiment of the previous aspects, the influenza virus is gamma-irradiated while frozen.

In one embodiment of the previous aspects, the influenza virus is strain A/PC.

In an additional embodiment of the previous aspects, the influenza virus comprises neuraminidase and hemagglutinin proteins from a strain other than A/PC.

In a further embodiment of the previous aspects, the neuraminidase and hemagglutinin proteins are from an H5N1 subtype influenza A virus.

In another embodiment of the previous aspects, the H5N1 subtype influenza A virus is HPAI A(H5N1) (bird flu).

In a further embodiment of the previous aspects, the neuraminidase and hemagglutinin proteins are from an H1N1 subtype influenza A virus.

In another embodiment of the previous aspects, the H1N1 subtype influenza A virus is pandemic H1N1/09 virus (swine flu).

In one embodiment of the previous aspects, the influenza virus is prepared by gamma-irradiating a frozen viral preparation.

In an additional embodiment of the previous aspects, the gamma-irradiated influenza virus is prepared by exposure of the influenza virus to a total dose of between about 6.5×10⁴ rad and about 2×10⁷ rad of gamma rays.

In an additional embodiment of the previous aspects, the gamma-irradiated influenza virus is prepared by exposure of the influenza virus to a total dose of about 1×10⁶ rad.

In a ninth aspect, the invention provides a cross-protective influenza vaccine produced by the method of the seventh aspect.

In one embodiment of the ninth aspect, the gamma-irradiated influenza virus is strain A/PC.

In one embodiment of the seventh, eighth and ninth aspects, the cross-protective influenza vaccine induces cross-protective cellular immunity.

In one embodiment of the seventh, eighth and ninth aspects, the cross-protective cellular immunity comprises either or both of:

(i) a cross-protective helper T-lymphocyte response

(ii) a cross-protective cytotoxic T-lymphocyte response.

In one embodiment of the seventh, eighth and ninth aspects, the cross-protective influenza vaccine induces cross-protective humoral immunity.

In another aspect the invention provides a method for preventing or treating an influenza virus infection in a subject, the method comprising administering to the subject a therapeutically effective amount of a gamma-irradiated influenza A subtype H3N2 virus.

In a further aspect, the invention provides a method for inducing or enhancing cross-protective immunity against multiple influenza virus subtypes in a subject, the method comprising administering to the subject a therapeutically effective amount of a gamma-irradiated influenza A subtype H3N2 virus.

### BRIEF DESCRIPTION OF THE DRAWINGS

A preferred embodiment of the present invention will now be described, by way of an example only, with reference to the accompanying drawings wherein:

FIG. 1 provides a series of graphs demonstrating superior lytic activity of immune cells from mice vaccinated with gamma-irradiated A/Port Chalmers/1/1973 (H3N2) (“A/PC”) in response to homologous and heterologous infection as determined by ⁵¹Cr release assays. Splenocytes from mice vaccinated with A/WSN (H1N1), A/PR8 (H1N1), A/JAP (H2N2), A/PC (H3N2) or A/X31 (H3N2) were co-cultured with NPP-labelled (column 1), A/PR8-infected (column 2), or A/PC-infected (column 3) stimulator cells and mock-infected (row 1), treated with NPP (row 2), infected with live A/PR8 (row 3) or infected with live A/PC (row 4).

FIG. 2 provides a series of graphs illustrating that intranasal vaccination with gamma-irradiated A/PC provides superior protection against heterotypic virus challenge compared to other routes of administration. Groups of 10 BALB/c mice were either mock treated (A) or vaccinated with gamma-irradiated A/PC (3.2×10⁶ PFU equivalent) intravenously (B) or intranasally (C). Mice were challenged intranasally after 3 weeks with a lethal dose (6×10² PFU) of A/PR8 and their weight recorded daily for 21 days. Survival after challenge with a lethal dose (6×10² PFU) of A/PR8 is defined by 30% weight loss (D) of mice mock treated, or vaccinated i.n., i.v., i.p., or s.c. and challenged as for (A-C) and monitored for 21 days.

FIG. 3 provides a series of graphs showing that gamma-irradiated A/PC influenza virus protects mice against both homologous and heterosubtypic challenge. (A, F)=mock treated; (B, G)=intranasally immunized with formalin inactivated A/PC; (C, H) intranasally immunized with UV inactivated A/PC; (D, I)=intranasally immunized with gamma-ray inactivated A/PC; (E, J)=survival after 20 days; *P<0.05 vs. control naïve group; Fisher's exact test.

FIG. 4 provides representative photomicrographs of immunohistochemically stained lung tissue following homologous challenge. (A)=naïve lung; (B)=unvaccinated (infected); (C)=gamma-A/PC vaccinated (challenged); (D)=formalin-A/PC vaccinated (challenged); E=UV-A/PC vaccinated (challenged).

FIG. 5 provides representative photomicrographs of immunohistochemically stained lung tissue following heterosubtypic challenge. (A)=naïve lung; (B)=unvaccinated (infected); (C)=gamma-A/PC vaccinated (challenged); (D)=formalin-A/PC vaccinated (challenged); E=UV-A/PC vaccinated (challenged).

FIG. 6 is a graph showing a comparison of Tc cell responses induced by live and inactivated A/PC. Mean values±SD of two mice per group are shown. Specific lysis values were interpolated from regression curves at effector:target ratio of 50:1. N.D.: not detected.

FIG. 7 provides a series of graphs illustrating that intranasal immunization with gamma-irradiated A/PC provides protection against high-dose A/PR8 lethal challenge. (A, C)=mice challenged with LD50 A/PR8; (B, D)=mice challenged with 5×LD50 A/PR8; (E)=mice challenged with 50×LD50 A/PR. (F)=survival and weight loss after 20 days. *P<0.05 vs. control naïve group; Fisher's exact test.

FIG. 8 provides a series of graphs illustrating that heterosubtypic protective properties of gamma-irradiated A/PC are maintained after a three-month period. (A)=mock treated; (B)=challenged with heterosubtypic strain A/PR8; (C)=survival and weight loss after 20 days in mice vaccinated with gamma-ray inactivated A/PC and naïve mice. *P<0.05 vs. control naïve group; Fisher's exact test.

FIG. 9 provides a series of graphs illustrating that heterosubtypic protective properties of gamma-irradiated A/PC are maintained after a freeze-drying process. (A)=mock treated; (B)=challenged with freeze-dried gamma-ray inactivated A/PR8; (C)=survival and weight loss after 20 days. *P<0.05 vs. control naïve group; Fisher's exact test.

FIG. 10 provides a series of graphs demonstrating that adoptive transfer of gamma-influenza-immune T-lymphocytes (but not B-lymphocytes) provides protection from heterosubtypic challenge. Immune splenic lymphocytes obtained from mice immunized i.v. with gamma-irradiated A/PC at 3 weeks post-immunization were enriched for T and B-lymphocytes. Recipient naïve mice received an i.v. transfer of enriched T-lymphocytes (A) or B-lymphocytes (B) or received no transfer (C). Three hours after cell transfer, mice were infected with A/PR8 and monitored for weight loss daily for 20 days.

### DEFINITIONS

As used in this application, the singular form “a”, “an” and “the” include plural references unless the context clearly dictates otherwise. For example, the term “a virus” also includes a plurality of viruses.

As used herein, the term “comprising” means “including.” Variations of the word “comprising”, such as “comprise” and “comprises”, have correspondingly varied meanings. Thus, for example, a polynucleotide “comprising” a sequence encoding a protein may consist exclusively of that sequence or may include one or more additional sequences.

As used herein, the phrases “cross-protective immune response” and “cross-protective immunity” are used interchangeably and have the same intended meaning. In the context of influenza virus infection each phrase refers to an immune response specific for multiple subtypes of influenza virus. The multiple subtypes may be of the same and/or different influenza types (i.e. influenza type A, influenza type B and/or influenza type C).

As used herein, a “cross-protective influenza vaccine” is a vaccine capable of inducing a “cross-protective immune response” in a subject to which it is administered.

As used herein, the term “backbone” in the context of an influenza virus encompasses a portion of that virus including all internal proteins present in an assembled mature virion and excluding all surface protein(s). For example, a strain A/PC “backbone” comprises all internal proteins present in assembled mature A/PC virus excluding the hemagglutinin (HA) and neuraminidase (NA) surface proteins.

As used herein, the terms “antibody” and “antibodies” include IgG (including IgG1, IgG2, IgG3, and IgG4), IgA (including IgA1 and IgA2), IgD, IgE, or IgM, and IgY, whole antibodies, including single-chain whole antibodies, and antigen-binding fragments thereof. Antigen-binding antibody fragments include, but are not limited to, Fab, Fab′ and F(ab′)2, Fd, single-chain Fvs (scFv), single-chain antibodies, disulfide-5 linked Fvs (sdFv) and fragments comprising either a VL or VH domain. The antibodies may be from any animal origin. Antigen-binding antibody fragments, including single-chain antibodies, may comprise the variable region(s) alone or in combination with the entire or partial of the following: hinge region, CH1, CH2, and CH3 domains. Also included are any combinations of variable region(s) and hinge region, CH1, CH2, and CH3 domains. Antibodies may be monoclonal, polyclonal, chimeric, multispecific, humanized, and human monoclonal and polyclonal antibodies which specifically bind the biological molecule.

It will be understood that use of the term “about” herein in reference to a recited numerical value (e.g. a dose of gamma-irradiation) includes the recited numerical value and numerical values within plus or minus ten percent of the recited numerical value.

It will be understood that use of the term “between” herein when referring to a range of numerical values encompasses the numerical values at each endpoint of the range. For example, a dose of gamma-irradiation between 1×10³ rad and 2×10⁹ rad is inclusive of the doses 1×10³ rad and 2×10⁹ rad.

Any description of prior art documents herein, or statements herein derived from or based on those documents, is not an admission that the documents or derived statements are part of the common general knowledge of the relevant art.

For the purposes of description all documents referred to herein are hereby incorporated by reference in their entirety unless otherwise stated.

### DETAILED DESCRIPTION OF THE INVENTION

Commercially-produced seasonal influenza vaccines suffer from a number of deficiencies, the most notable being an inability to induce cross-protective immunity. Hence, they are effective only against the specific influenza strains on which they are based and offer almost no protection against other circulating strains. In addition, seasonal influenza vaccines provide little or no protection against newly emerging strains which are generally responsible for influenza epidemics and pandemics. The present reliance on seasonal flu vaccines also means that vaccines for newly emerging strains cannot be stockpiled and hence in many cases flu epidemics run their full course before a suitable vaccine can be generated.

The inability of seasonal flu vaccines to provide cross-protective immunity is believed to arise at least in part from a failure to induce significant levels of antiviral T-lymphocyte responses. T-lymphocyte responses against influenza viruses are predominantly directed against internal viral proteins which are highly conserved among all influenza virus subtypes and less susceptible to mutation. Hence, vaccines that induce T-lymphocyte responses are more likely to confer cross-protective immunity to both existing and newly arising influenza strains.

The present inventors have determined that treatment of influenza viruses with gamma-irradiation provides a means of inactivating the virus while preserving the antigenicity of both internal and external viral proteins. The preservation of viral proteins obtained by inactivating with gamma-irradiation is believed to significantly enhance T-lymphocyte-mediated responses against antigenic determinants of internal viral proteins leading to the generation of cross-protective immunity. Surprisingly, the present inventors have demonstrated that gamma-irradiated A/Port Chalmers/1/1973 (H3N2) virus (“A/PC”) induces stronger cross-protective T-lymphocyte responses against influenza viruses than other gamma-irradiated influenza strains. Without limitation to a particular mode of action, comparative experimental data provided in the Examples of the present specification suggests that the backbone of gamma-irradiated A/PC (i.e. the portion containing only internal viral proteins) may be responsible for inducing the strong cross-protective T-lymphocyte responses observed. It is postulated that backbone protein(s) (e.g. the nucleoprotein and/or matrix protein(s)) of gamma-irradiated A/PC may be more accessible to the host immune system than those of other gamma-irradiated influenza strains, leading to the induction of stronger cross-protective T-lymphocyte responses by gamma-irradiated A/PC.

The accessibility of internal backbone proteins to the host immune system may also be aided by the particular hemagglutinin (HA) and neuraminidase (NA) surface proteins of gamma-irradiated A/PC (H3N2). These are suggested to bind more efficiently to target T-lymphocyte receptors compared to HA/NA proteins of other gamma-irradiated virus strains. This may enhance the capacity of gamma-irradiated A/PC to enter host cells where internal backbone proteins can be processed and displayed for recognition by T-lymphocytes.

Accordingly, certain aspects of the invention relate to gamma-irradiated influenza virus for preventing and treating influenza infection. The invention also provides gamma-irradiated influenza virus for inducing or enhancing cross-protective immunity against multiple influenza virus subtypes. The gamma-irradiated virus comprises a backbone of strain A/PC internal proteins. The gamma-irradiated virus may additionally comprise surface protein(s) (e.g. hemagglutinin and/or neuraminidase) derived from any influenza virus subtype(s). Accordingly, a gamma-irradiated influenza virus of the invention may be a gamma-irradiated A/PC virus or alternatively a recombinant gamma-irradiated virus comprising an A/PC backbone in combination with surface protein(s) derived from different influenza strain(s).

Additional aspects of the invention relate to methods for treating and preventing influenza virus infection. Also provided are methods for inducing or enhancing cross-protective immunity against multiple influenza virus subtypes. The methods comprise administering to a subject in need thereof a gamma-irradiated influenza virus of the invention as described in the paragraph directly above. In certain embodiments the virus administered is gamma-irradiated A/PC.

Further aspects of the invention relate to methods for producing a cross-protective influenza vaccine capable of inducing an immune response effective against multiple subtypes of influenza virus. The methods comprise inactivating a preparation of influenza A virus comprising a backbone of strain A/PC internal proteins by gamma-irradiation. The virus may additionally comprise surface protein(s) (e.g. hemagglutinin and/or neuraminidase) derived from any influenza virus subtype(s). In certain embodiments the virus inactivated by gamma-irradiation is strain A/PC.

### Gamma-Irradiated Influenza Viruses

### A/PC Backbone

Gamma-irradiated influenza viruses of the invention may comprise a backbone of strain A/Port Chalmers/1/1973 (also referred to herein as strain “A/PC”) internal proteins. The viruses may additionally comprise surface protein(s) (e.g. hemagglutinin, neuraminidase and/or hemagglutinin-esterase fusion) derived from any influenza virus subtype(s).

The skilled addressee will recognise that influenza strain A/Port Chalmers/1/1973 (H3N2) is also known by a number of related names, including, for example, A/Port Chalmers/73(H3N2), A/Port Chalmers/1/1973(H3N2), A/Port Chalmers/1/73(H3N2), A/Port Chalmers/1973(H3N2), and STRAIN A/PORT CHALMERS/1/73. In general, influenza strain A/PC as contemplated herein may be that identified by UniProt taxon identifier 385624 (see http://www.uniprot.org/).

A “backbone” of strain A/PC proteins as contemplated herein encompass all internal proteins present in an assembled mature strain A/PC virion but excludes all surface proteins (i.e. hemagglutinin (HA) and neuraminidase (NA)).

Accordingly, a backbone of strain A/PC proteins as contemplated herein comprises each of the following A/PC strain internal proteins: matrix protein 1 (M1), matrix protein 2 (M2), nuclear export protein (NEP) (also known as non-structural protein 2 (NS2)), polymerase acidic protein (PA), polymerase basic protein 1 (PB 1) (also known as RNA-directed RNA polymerase catalytic subunit and RNA-directed RNA polymerase subunit P1), polymerase basic protein 2 (PB2), and nucleoprotein (NP); and excludes the following A/PC strain surface proteins: hemagglutinin (HA) and neuraminidase (NA).

A backbone of strain A/PC proteins therefore encompasses internal viral proteins encoded by gene segments (i.e. PB1, PB2, M, NP, NS, and PA) with the exception of non-structural protein 1 (NS1) and protein PBF1-F2 which are generally expressed only during active infection and not present in assembled mature A/PC virions.

Non-limiting examples of a strain A/PC backbone protein sequences are provided under UniProt accession number P63234 (matrix protein 1), UniProt accession number P63232 (matrix protein 2), UniProt accession number Q1PUD4 (nuclear export protein), UniProt accession number Q1PUD2 (polymerase acidic protein), UniProt accession number Q1PUD1 (polymerase basic protein 1), UniProt accession number Q1PUC9 (polymerase basic protein 2), and UniProt accession number Q1PUD5 (nucleoprotein).

Accordingly, a gamma-irradiated influenza virus of the invention may be a gamma-irradiated A/PC virus or alternatively a recombinant gamma-irradiated virus comprising an A/PC backbone in combination with surface protein(s) derived from different influenza strain(s).

A recombinant gamma-irradiated virus of the invention may comprise surface protein(s) from any influenza virus subtype, including influenza type A (HA and NA), influenza type B (HA and NA) or influenza type C (HEF) surface protein(s), and combinations thereof.

Preferred combinations of surface proteins from influenza virus A subtypes include, but are not limited to, H1N1 (e.g. H1N1 09 Swine Flu; A/California/07/2009 lineage viruses; A/Brisbane/59/2007 lineage viruses), H1N2, H1N7, H2N2, H3N1, H3N2 (e.g. A/Brisbane/10/2007 lineage viruses, A/Perth/16/2009 lineage viruses), H3N8, H4N8, H5N1 (e.g. HPAI A(H5N1)), H5N2, H5N3, H5N8, H5N9, H6N5, H7N1, H7N2, H7N3, H7N4, H7N7, H8N4, H9N2, H10N7, H11N6, H12N5, H13N6, H14N5, and recombinants thereof.

In certain embodiments, gamma-irradiated viruses of the invention comprise H3N2 surface protein(s). Preferably, the H3N2 surface proteins are derived from strain A/PC. Accordingly, gamma-irradiated viruses of the invention may be gamma-irradiated A/PC viruses.

In other embodiments, gamma-irradiated viruses of the invention comprise H5N1 surface protein(s). Preferably, the H5N1 surface proteins are derived from HPAI A(H5N1) (bird flu).

In additional embodiments, gamma-irradiated viruses of the invention comprise H1N1 surface protein(s). Preferably, the H1N1 surface proteins are derived from pandemic H1N1/09 virus (swine flu).

Preferred surface proteins from influenza B viruses include, but are not limited to, those derived from strains of the B/Victoria lineage (e.g. B/Victoria/02/87 viruses), strains of the B Yamagata lineage (e.g. B/Yamagata/16/88 viruses), and strains of the B/Brisbane lineage (e.g. B/Brisbane/60/2008 lineage viruses).

Preferred surface proteins from influenza virus C viruses include, but are not limited to, those derived from strains of the Kanagawa/1/76 (KA/1/76), Yamagata/26/81 (YA/26/ 81), Aichi/1/81 (AI/1/81), SP/378/82, and Mississippi/80 (MS/80) lineages.

Compositions and vaccines of the invention may comprise mixtures of two or more different gamma-irradiated viruses of the invention (e.g. one or more A/PC viruses in combination with one or more recombinant viruses comprising an A/PC backbone; or two or more recombinant viruses comprising an A/PC backbone).

### Production And Purification of Influenza Viruses

In certain embodiments, gamma-irradiated influenza viruses of the invention comprise an A/PC strain backbone in combination with surface proteins derived from other influenza strain(s) or subtype(s) (i.e. recombinant influenza viruses).

Methods for the generation of recombinant influenza viruses are known in the art. For example, recombinant influenza viruses may be produced using standard plasmid-based reverse genetics techniques (see, for example, Luytjes, et al., (1989), “Amplification, expression, and packaging of a foreign gene by influenza virus”, Cell, 59:1107-1113; Enami et al., (1990), “Introduction of site specific mutations into the genome of influenza virus”, Proc. Natl. Acad. Sci. USA 87:3802-3805; Fodor et al., (1999), “Rescue of Influenza A Virus from Recombinant DNA”, J. Virol., 73 (11):9679-82; Quinlivan et al., (2005), “Attenuation of equine influenza viruses through truncations of the NS1 protein”, J. Virol., 79:8431-9; and Gao et al., (2008), “A Seven-Segmented Influenza A Virus Expressing the Influenza C Virus Glycoprotein HEF”, J. Virol., 82 (13):6419-6426).

Another method for generating chimeric influenza virus-like particles is described in U.S. Pat. No. 7,556,940.

A/PC strain and recombinant influenza viruses of the invention can be propagated using methods known in the art. For example, the viruses may be propagated by serial passaging in embryonated eggs as described, for example, in Coico et al., (Eds), (2000-2010), “Current Protocols in Microbiology”, John Wiley and Sons, Inc. (see in particular Unit 15G.1 entitled “Influenza: Propagation, Quantification, and Storage”) and/or propagated in cell culture (see, for example, Furminger, “Vaccine Production”, in Nicholson et al., (Eds), (1998), “Textbook of Influenza”, Chapter 24, pp. 324-332, London Blackwell Scientific Publications; Merten et al., (1996), “Production of influenza virus in cell cultures for vaccine preparation”, in Cohen and Shafferman (Eds), (1996), “Novel Strategies in Design and Production of Vaccines”, pp. 141-151; U.S. Pat. No. 5,698,433; U.S. Pat. No. 5,753,489; U.S. Pat. No. 5,824,536; U.S. Pat. No. 6,146,873; U.S. Pat. No. 6,344,354; U.S. Pat. No. 6,455,298; and U.S. Pat. No. 6,951,752).

Influenza virus propagated using the methods above (or by any other means) may be purified and/or concentrated prior to gamma-irradiation. Any suitable method known in the art may be used for this purpose. For example, influenza virus may be purified by temperature-dependent adsorption to chicken red blood cells using the method described by Laver, (1969), “Purification of influenza virus”, in Habel and Salzman (Eds), (1969), “Fundamental Techniques in Virology”, pp. 82-86, Academic Press, New York.

In preferred embodiments, influenza virus is purified and/or concentrated prior to gamma-irradiation using tangential/cross-flow filtration. For example, virus-containing fluid may be applied to a filtering device such as a membrane having an appropriate pore size (e.g. less than about 80 nm). The fluid may be pumped tangentially along the surface of the membrane (i.e. across the surface) and pressure applied to force a portion of the fluid through the membrane to the filtrate side. The applied pressure will generally be of a degree that does not adversely affect virion structure and/or the integrity of viral antigens. Filtrate containing viral particles passes through the membrane, whereas particulates and macromolecules in the fluid that are too large to pass through the membrane pores are predominantly retained on the opposing side. In general, retentate (i.e. retained components) does not build up at the surface of the membrane and may instead be swept along by the tangential flow. The retentate may be re-diluted with appropriate media (e.g. PBS containing dextran and/or sucrose) and the filtration process repeated if required. The use of tangential/cross-flow filtration to purify influenza virus used for gamma-irradiation provides an advantage over purification techniques currently used for influenza vaccine preparation (e.g. ultracentrifugation) as the integrity of viral antigens is better preserved. This in turn enhances the immunogenicity of gamma-irradiated viral preparations, and in particular their ability to elicit cross-protective immunity against multiple influenza subtypes and strains.

### Gamma-Irradiation

Influenza virus for use in the methods, compositions and vaccines of the invention is gamma-irradiated. Any suitable source of gamma-radiation may be used. Convenient gamma emitters include, but are not limited to, Ba¹³⁷, Co⁶⁰, Cs¹³⁷, Ir¹⁹², U²³⁵, Se⁷⁵ and Yb¹⁶⁹.

Gamma-irradiation of influenza virus may be performed using commercially available devices such as, for example, a Gammacell irradiator manufactured by Atomic Energy of Canada Ltd., Canada (e.g. Gammacell 40 Irradiator, Gammacell 220 Irradiator, Gammacell 1000 irradiator, Gammacell 3000 irradiator), a gamma-irradiator manufactured by J. L. Shepherd and Associates (San Fernando, Calif., USA), or a Nordion Gamma Cell-1000 irradiator manufactured by Nordion Inc. (Kanata, Ontario, Canada). Other suitable devices are described, for example, in U.S. Pat. No. 3,557,370 and U.S. Pat. No. 3,567,938.

Preferably, the influenza virus is exposed to a dose of gamma-irradiation sufficient to inactivate it. More preferably, the dose of gamma-irradiation is sufficient to inactivate the virus without substantially disrupting the structure of viral proteins thus retaining the immunogenicity of antigenic determinants. Hence, in a preferred embodiment of the invention, influenza virus is treated with a dose of gamma-irradiation capable of inactivating the virus while retaining its antigenic structure. Preferably, the dose of gamma-irradiation is administered to the virus over a period of time and at a level sufficient to ensure that all viruses under treatment are exposed and inactivated without adversely affecting the integrity of viral antigenic determinants.

For example, influenza virus may be exposed to a total dose of gamma-irradiation of between about 1×10³ rad and about 2×10⁹ rad. In certain embodiments of the invention, influenza virus is exposed to a total dose of gamma-irradiation of between about 1×10³ rad and about 2×10⁹ rad. Preferably, the influenza virus is exposed to a total dose of gamma-irradiation of between about 6.5×10⁴ rad and about 2×10⁷ rad. More preferably, the influenza virus is exposed to a total dose of gamma-irradiation of about 1×10⁶ rad.

The optimal dose of gamma-irradiation may be influenced by factors such as the medium in which the virus is present, the amount of virus to be treated, the temperature of the virus present, and/or the subtype or strain of virus under treatment. Accordingly, the total dose of gamma-irradiation, the exposure time and/or the level of gamma-irradiation applied over the period of exposure may be optimised to enhance the effectiveness of the treatment.

The total dose of gamma-irradiation may be administered to the virus cumulatively over a period of time. For example, gamma-irradiation may be administered to the virus at a level lower than that of the total dose, over a time period sufficient to achieve the total dose of gamma-irradiation required.

In preferred embodiments, influenza virus preparations are maintained in a frozen state while being exposed to gamma-irradiation. This may facilitate the preservation of biological integrity and avoid damage of viral antigens thereby enhancing the immunogenicity of gamma-irradiated viral preparations, and in particular, their ability to elicit cross-protective immunity against multiple influenza subtypes and strains. In general, a gamma-irradiation dose of between about 10 kGy and about 20kGy may be effective for treating frozen viral preparations.

As mentioned above, it is preferable that treatment with gamma-irradiation is sufficient to inactivate the influenza virus without substantially disrupting the structure of viral antigens. Inactivation of the virus may be assessed using methods generally known in the art. For example, viral infectivity can be measured following gamma-irradiation by inoculating embryonic eggs and/or cell lines as described above to determine whether the virus is capable of propagation.

The integrity of antigenic determinants can be assessed, for example, by assaying the virus for hemagglutinating activity following gamma-irradiation. Methods for performing hemagglutination assays are known in the art and are described, for example, in Coico et al., (Eds), (2000-2010), “Current Protocols in Microbiology”, John Wiley and Sons, Inc. (see in particular Unit 15G.1 entitled “Influenza: Propagation, Quantification, and Storage”); and Sato et al., (1983), “Separation and purification of the hemagglutinins from Bordetella pertussis”, Infect. Immun., 41, 313-320.

Additionally or alternatively, a neuraminidase assay may be used to assess the integrity of viral antigenic determinants (see, for example, Khorlin et al., (1970), “Synthetic inhibitors of Vibrio cholerae neuraminidase and neuraminidases of some influenza virus strains”, FEBS Lett., 8:17-19; and Van Deusen et al., (1983), “Micro neuraminidase-inhibition assay for classification of influenza A virus neuraminidases”, Avian Dis., 27:745-50).

Additionally or alternatively, the detection of cytotoxic T-lymphocyte responses against viral proteins induced by the gamma-irradiated preparations may be detected, for example, by ⁵¹Cr release assay as described in the Examples of the present specification.

### Prevention And Treatment of Influenza Infection

The present invention provides methods for preventing and treating influenza virus infection in a subject. The methods comprise administering to the subject a therapeutically effective amount of a gamma-irradiated influenza virus of the invention (see section above entitled “Gamma-irradiated influenza vaccines”).

Methods suitable for the inactivation of virus preparations by gamma-irradiation are described in the section above entitled “Gamma-irradiation”.

The gamma-irradiated influenza viruses may be administered to the subject in the form of a composition or vaccine of the invention (see sections below entitled “Pharmaceutical compositions” and “Cross protective influenza vaccines”).

The “subject” may be any animal of economic, social or research importance including bovine, equine, ovine, primate (including human and non-human primates), avian (e.g. poultry including chickens, turkeys, pheasants, ducks, geese, quails, guineafowl, peafowl, ostriches, pigeons, and doves) and rodent species. In certain embodiments, the subject is a mammal. The mammal may be a human. The subject may be at risk of infection with influenza virus, infected with influenza virus, suspected of infection with influenza virus, and/or previously infected with influenza virus.

The term “therapeutically effective amount” as used herein, includes within its meaning a non-toxic but sufficient amount of gamma-irradiated influenza viruses of the invention (or compositions or vaccines comprising the same) to provide the desired therapeutic effect. The exact amount required will vary from subject to subject depending on factors such as the viral subtype/strain being treated, the age and general condition of the subject, the severity of the condition being treated, the particular agent being administered, the mode of administration, and so forth. Thus, it is not possible to specify an exact “effective amount”. However, for any given case, an appropriate “effective amount” may be determined by one of ordinary skill in the art using only routine experimentation.

In general, a “therapeutically effective amount” of a gamma-irradiated influenza virus of the invention (or a composition or vaccine comprising the same) is capable of inducing and/or enhancing an immune response against multiple strains and subtypes of influenza virus. Preferably, the immune response is induced and/or enhanced against strains from one or more different types of influenza. Typically, a therapeutically effective amount when administered to a subject will induce an immune response sufficient to prevent infection, diminish the severity of infection upon subsequent exposure of said subject to influenza virus, and/or diminish one or more symptoms of an influenza virus infection. It will be understood that reduction in any one or more symptoms typically seen in influenza infection is encompassed within the meaning, for example a decrease in the duration of infection, and/or a decrease in the duration of one or more symptoms, such as fever, headache, cough, painful throat, body aches, muscle pain, nasal congestion, coughing, sneezing, reddened eyes, skin, mouth, throat and nose, diarrhea, vomiting and fatigue.

Accordingly, the invention provides methods for the prevention and treatment of influenza virus infection by administration of a therapeutically effective amount of gamma-irradiated influenza viruses of the invention. The administration of mixtures of two or more different gamma-irradiated influenza viruses of the invention (e.g. one or more gamma-irradiated A/PC viruses in combination with one or more recombinant viruses comprising an A/PC backbone; two or more recombinant viruses comprising an A/PC backbone) is also contemplated.

In certain embodiments, the methods of preventing or treating influenza virus infection comprise administering a therapeutically effective amount of a gamma-irradiated A/PC influenza virus to the subject.

In other embodiments, the methods comprise administering a therapeutically effective amount of a gamma-irradiated recombinant influenza virus to the subject. The recombinant virus may comprise an A/PC backbone in combination with H5N1 surface proteins derived from HPAI A(H5N1) (bird flu). Alternatively, the recombinant virus may comprise an A/PC backbone in combination with H1N1 surface proteins derived from pandemic H1N1/09 virus (swine flu).

The gamma-irradiated viruses of the invention (or compositions and vaccines comprising the same) may be administered to the subject by any suitable route (see section below entitled “Routes of administration”). Preferably, the gamma-irradiated influenza viruses are administered by the intranasal route. The gamma-irradiated influenza viruses may be prepared in a freeze-dried form (see Section below entitled “Cross protective influenza vaccines”). The gamma-irradiated influenza viruses may be prepared from a virus stock purified by tangential/cross-flow filtration as described in the subsection above entitled “Production and purification of influenza viruses”. Viral stock used to prepare gamma-irradiated influenza viruses administered to the subject may be frozen during gamma-irradiation (see subsection above entitled “Production and purification of influenza viruses”).

### Cross-Protective Immunity

The invention provides methods for inducing or enhancing cross-protective immune responses against multiple influenza virus strains and subtypes in a subject. The method comprises administering to the subject a therapeutically effective amount of a gamma-irradiated influenza virus of the invention (see section above entitled “Gamma-irradiated influenza viruses”).

Methods suitable for the inactivation of the virus preparations by gamma-irradiation are described in the section above entitled “Gamma-irradiation”.

The gamma-irradiated influenza virus may be administered to the subject in the form of a composition or vaccine of the invention (see sections below entitled “Pharmaceutical compositions” and “Cross protective influenza vaccines”).

The “subject” may be any animal of economic, social or research importance including bovine, equine, ovine, primate (including human and non-human primates), avian (e.g. poultry including chickens, turkeys, pheasants, ducks, geese, quails, guineafowl, peafowl, ostriches, pigeons, and doves) and rodent species. In certain embodiments, the subject is a mammal. The mammal may be a human. The subject may be at risk of infection with influenza virus, infected with influenza virus, suspected of infection with influenza virus, and/or previously infected with influenza virus.

“Cross-protective immunity” as contemplated herein refers to an immune response specific for multiple subtypes and strains of influenza virus. The multiple subtypes and strains may be of the same and/or different influenza types (i.e. influenza type A, influenza type B and/or influenza type C).

Preferably, the methods of the invention induce or enhance cross-protective immunity against conserved influenza virus antigens. For example, cross-protective immunity may be induced or enhanced against conserved influenza virus antigens that are not subject to antigenic shift and/or drift, or influenza virus antigens that are subject to a negligible degree of antigenic shift and/or drift. A negligible degree of antigenic shift and/or drift will, in general, be a degree of antigenic shift and/or drift that does not alter the ability of host immune cells to recognise and respond to the antigen.

In general, cross-protective immune responses induced or enhanced by the methods of the invention may be directed towards influenza virus antigen(s) that are identical or substantially similar in one or more different influenza virus subtypes or strains. A cross-protective immune cell will thus recognise and respond to substantially similar or identical viral antigen(s) shared by multiple virus subtypes and strains.

The methods of the invention may be utilised to induce or enhance cross-protective cellular immunity against influenza viruses. Cross-protective cellular immunity may comprise, for example, cross-protective helper T-lymphocyte responses and/or cross-protective cytotoxic T-lymphocyte responses. Preferably, cross-protective cellular immunity will involve cross-protective cytotoxic CD8⁺T-lymphocyte responses. The induction or enhancement of the cross-protective cellular immunity can be detected using methods known in the art. For example, cross-protective T-lymphocytes (e.g. helper T-lymphocytes and cytotoxic T-lymphocytes) may be detected or quantified using methods commonly used for the detection virus-specific T-lymphocytes such as ELISpot assays, intracellular cytokine staining assays and tetramer-based assays.

Additionally or alternatively, the methods of the invention may be utilised to induce or enhance cross-protective humoral immunity against influenza viruses. The cross-protective humoral immunity may comprise cross-protective B-lymphocyte responses and an increased quantity of cross-protective influenza virus-specific antibodies (e.g. IgA and IgG) in the circulation of the subject. In general, a cross-protective antibody will have the ability to interact with an antigen from an influenza virus strain or subtype that did not stimulate its production. Cross-protective B-lymphocytes and antibodies may be detected using methods known in the art (e.g. by microneutralisation assay, flow cytometry or immunohistochemistry). Specific examples of suitable techniques for detecting cross-protective influenza virus-specific antibodies are described in, for example, Rota et al., (1987), “Comparison of the immune response to variant influenza type B hemagglutinins expressed in vaccinia virus”, Virology, 161:269-75; and Harmon et al., (1988), “Antibody Response in Humans to Influenza Virus Type B Host-Cell-Derived Variants after Vaccination with Standard (Egg-Derived)Vaccine or Natural Infection”, J. Clin. Microbiol., 26:333-337.

In certain embodiments, the methods for inducing or enhancing cross-protective immune responses against multiple influenza virus strains and subtypes in a subject comprise administering a therapeutically effective amount of a gamma-irradiated A/PC influenza virus to the subject.

In other embodiments, the methods comprise administering a therapeutically effective amount of a gamma-irradiated recombinant influenza virus to the subject. The recombinant virus may comprise an A/PC backbone in combination with H5N1 surface proteins derived from HPAI A(H5N1) (bird flu). Alternatively, the recombinant virus may comprise an A/PC backbone in combination with H1N1 surface proteins derived from pandemic H1N1/09 virus (swine flu).

The gamma-irradiated viruses of the invention (or compositions and vaccines comprising the same) may be administered to the subject by any suitable route (see section below entitled “Routes of administration”). Preferably, the gamma-irradiated influenza viruses are administered by the intranasal route. The gamma-irradiated influenza viruses may be prepared in a freeze-dried form (see Section below entitled “Cross protective influenza vaccines”). The gamma-irradiated influenza viruses may be prepared from a virus stock purified by tangential/cross-flow filtration as described in the subsection above entitled “Production and purification of influenza viruses”. Viral stock used to prepare gamma-irradiated influenza viruses administered to the subject may be frozen during gamma-irradiation (see subsection above entitled “Production and purification of influenza viruses”).

### Pharmaceutical Compositions

The present invention provides compositions comprising gamma-irradiated influenza viruses of the invention. Non-limiting examples of suitable gamma-irradiated influenza viruses and methods for their preparation are described above in the section above entitled “Gamma-irradiated influenza viruses”.

In certain embodiments, compositions of the present invention are pharmaceutical compositions, non-limiting examples of which include preventative and/or therapeutic vaccines. Pharmaceutical compositions of the present invention may be prepared using methods known to those of ordinary skill in the art. Non-limiting examples of suitable methods are described in Gennaro et al. (Eds), (1990), “Remington's Pharmaceutical Sciences”, Mack Publishing Co., Easton, Pa., USA.

A composition of the invention may be administered to a recipient in isolation or in combination with other additional therapeutic agent(s). In embodiments where the composition is administered with therapeutic agent(s), the administration may be simultaneous or sequential (i.e. composition administration followed by administration of the agent(s) or vice versa).

Compositions of the present invention may comprise a pharmaceutically acceptable carrier, excipient, diluent and/or adjuvant. “Pharmaceutically acceptable” carriers, excipients, diluents and/or adjuvants as contemplated herein are substances which do not produce adverse reaction(s) when administered to a particular recipient such as a human or non-human animal. Pharmaceutically acceptable carriers, excipients, diluents and adjuvants are generally also compatible with other ingredients of the composition. Non-limiting examples of suitable excipients, diluents, and carriers can be found in Rowe et al. (Eds), (2003), “Handbook of Pharmaceutical Excipients” 4th Edition, The Pharmaceutical Press, London, American Pharmaceutical Association, Washington.

Non-limiting examples of pharmaceutically acceptable carriers, excipients or diluents include demineralised or distilled water; saline solution; vegetable based oils such as peanut oil, safflower oil, olive oil, cottonseed oil, maize oil, sesame oils, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or isopropanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

Compositions of the invention can be administered to a recipient by standard routes, including, but not limited to, parenteral (e.g., intravenous, intraspinal, subcutaneous or intramuscular), oral, mucosal (e.g. intranasal) or topical routes.

Accordingly, compositions of the invention may be in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form suitable for administration by inhalation, such as by intranasal inhalation or oral inhalation, or in a form suitable for parenteral administration, that is, subcutaneous, intramuscular or intravenous injection.

Solid forms of compositions of the invention for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms of compositions of the invention for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof

Suspensions comprising compositions of the invention for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

For preparation of compositions as injectable solutions or suspensions, non-toxic parenterally acceptable diluents or carriers may be used such as Ringer's solution, isotonic saline, phosphate buffered saline, ethanol and 1,2 propylene glycol.

Emulsions for oral administration may further comprise one or more emulsifying agents. Suitable emulsifying agents include dispersing agents as exemplified above or natural gums such as guar gum, gum acacia or gum tragacanth.

Topical formulations of the invention comprise an active ingredient(s) (e.g. gamma-irradiated influenza viruses of the invention) together with one or more acceptable carriers, and optionally any other therapeutic ingredients. Formulations suitable for topical administration include liquid or semi-liquid preparations suitable for penetration through the skin to the site of where treatment is required, such as liniments, lotions, creams, ointments or pastes, and drops suitable for administration to the eye, ear or nose.

Drops according to the invention may comprise sterile aqueous or oily solutions or suspensions. These may be prepared by dissolving the active ingredient in an aqueous solution of a bactericidal and/or fungicidal agent and/or any other suitable preservative, and optionally including a surface active agent. The resulting solution may then be clarified by filtration, transferred to a suitable container and sterilised. For example, sterilisation may be achieved by filtration followed by transfer to a container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the invention include those suitable for application to the skin or eye. An eye lotion may comprise a sterile aqueous solution optionally containing a bactericide and may be prepared by methods similar to those described above in relation to the preparation of drops. Lotions or liniments for application to the skin may also include an agent to hasten drying and to cool the skin, such as an alcohol or acetone, and/or a moisturiser such as glycerol, or oil such as castor oil or arachis oil.

Creams, ointments or pastes according to the invention are semi-solid formulations of the active ingredient for external application. They may be made by mixing the active ingredient in finely-divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or polyethylene glycol (i.e. macrogol).

Compositions of the invention may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

Supplementary active ingredients, such as adjuvants or biological response modifiers, can also be incorporated into compositions of the invention.

Preferably, an adjuvant will enhance the immune response induced and/or enhanced by component(s) of a given composition thereby improving protective efficacy. Preferably, the adjuvant will enable the induction of protective immunity utilising a lower dose of other active component(s) (e.g. gamma-irradiated influenza viruses of the invention).

Any suitable adjuvant may be included in a composition of the invention. For example, an aluminium-based adjuvant may be utilised. Suitable aluminium-based adjuvants include, but are not limited to, aluminium hydroxide, aluminium phosphate and combinations thereof. Other specific examples of aluminium-based adjuvants that may be utilised are described in European Patent No. 1,216,053 and U.S. Pat. No. 6,372,223.

Oil in water emulsions may be utilised as adjuvants in compositions of the invention. Oil in water emulsions are well known in the art. In general, the oil in water emulsion will comprise a metabolisable oil, for example, a fish oil, a vegetable oil, or a synthetic oil. Examples of suitable oil in water emulsions include those described in European Patent No. 0399843, U.S. Pat. No. 7,029,678 and PCT Publication No. WO 2007/006939. The oil in water emulsion may be utilised in combination with other adjuvants and/or immunostimulants.

Non-limiting examples of other suitable adjuvants include immunostimulants such as granulocyte-macrophage colony-stimulating factor (GM-CSF), monophosphoryl lipid A (MPL), cholera toxin (CT) or its constituent subunit, heat labile enterotoxin (LT) or its constituent subunit, toll-like receptor ligand adjuvants such as lipopolysaccharide (LPS) and derivatives thereof (e.g. monophosphoryl lipid A and 3-Deacylated monophosphoryl lipid A), muramyl dipeptide (MDP) and F protein of Respiratory Syncytial Virus (RSV).

Adjuvants in compositions of the invention typically include emollients, emulsifiers, thickening agents, preservatives, bactericides and buffering agents. Another type of “self adjuvant” is provided by the conjugation of immunogenic peptides to lipids such as the water soluble lipopeptides Pam3Cys or its dipalmitoyl derivative Pam2Cys.

Such adjuvants have the advantage of accompanying and immunogenic component into the antigen presenting cell (such as dendritic cells) and thus producing enhanced antigen presentation and activation of the cell at the same time. These agents act at least partly through toll-like receptor 2 (see, for example, Brown and Jackson, (2005), “Lipid based self adjuvanting vaccines”, Current Drug Delivery, 23:83).

Suitable adjuvants are commercially available such as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminium salts such as aluminium hydroxide gel (alum) or aluminium phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

### Cross-Protective Influenza Vaccines

The present invention provides cross-protective influenza vaccines comprising gamma-irradiated influenza viruses of the invention. Non-limiting examples of suitable gamma-irradiated influenza viruses are provided above in the section entitled “Gamma-irradiated influenza viruses”.

Methods are also provided for the production of cross-protective influenza vaccines of the invention. The methods comprise inactivating a preparation of influenza virus of the invention by gamma-irradiation. Suitable examples of influenza virus preparations for use in the vaccine production methods of the invention are provided above in the section entitled “Gamma-irradiated influenza viruses”. Methods suitable for the inactivation of the virus preparations by gamma-irradiation are described in the subsection above entitled “Gamma-irradiation”.

In certain embodiments, the methods for the production of cross-protective influenza vaccines may comprise inactivating A/PC strain viruses by gamma-irradiation.

In other embodiments, the methods for the production of cross-protective influenza vaccines may comprise inactivating recombinant influenza viruses by gamma-irradiation. The recombinant viruses may comprise an A/PC backbone in combination with H5N1 surface proteins derived from HPAI A(H5N1) (bird flu). Alternatively, the recombinant viruses may comprise an A/PC backbone in combination with H1N1 surface proteins derived from pandemic H1N1/09 virus (swine flu).

Preferably, the cross-protective influenza vaccines are formulated for intranasal administration. The vaccines may be prepared in a freeze-dried form (see paragraphs below in this section) and/or prepared from a virus stock purified by tangential/cross-flow filtration as described in the subsection above entitled “Production and purification of influenza viruses”. Viral stock used to prepare vaccines of the invention may be frozen during gamma-irradiation (see subsection above entitled “Production and purification of influenza viruses”).

Vaccines of the invention may be administered to naïve recipients (i.e. individuals seronegative for particular target strain(s) of influenza) or primed recipients (i.e. individuals seropositive for particular target strain(s) of influenza).

Vaccines of the invention include both preventative vaccines (i.e. vaccines administered for the purpose of preventing influenza infection) and therapeutic vaccines (i.e. vaccines administered for the purpose of treating influenza infection). A vaccine of the invention may therefore be administered to a recipient for prophylactic, ameliorative, palliative, or therapeutic purposes.

Vaccines of the invention may be prepared according to standard methods known to those of ordinary skill in the art. Methods for vaccine preparation are generally described in Voller et al., (1978), “New Trends and Developments in Vaccines”, University Park Press, Baltimore, Md., USA.

Non-limiting examples of suitable pharmaceutically acceptable excipients, diluents, carriers and adjuvants that may be included in vaccines of the invention are provided in the subsection above entitled “Pharmaceutical compositions”.

In general, adjuvant activity in the context of a vaccine of the invention includes, but is not limited to, the ability to enhance the immune response (quantitatively or qualitatively) induced by immunogenic components in the vaccine (e.g. gamma-irradiated influenza viruses of the invention). This may reduce the dose or level of the immunogenic components required to produce an immune response and/or reduce the number or the is frequency of immunisations required to produce the desired immune response.

Although adjuvant(s) may be included in the vaccines, it is believed that similar levels of immunogenicity may be obtained using gamma-inactivated influenza viruses of the invention without an adjuvant. Hence, vaccines of the invention need not necessarily comprise an adjuvant. In such cases, reactogenicity problems arising from the use of adjuvants may be avoided.

Vaccines of the invention can be administered to a recipient by standard routes, including, but not limited to, parenteral (e.g. intravenous, intraspinal, subcutaneous or intramuscular), oral, topical, or mucosal routes (e.g. intranasal).

Preferably, vaccines of the invention are administered by the mucosal route. Non-limiting examples of acceptable routes of mucosal vaccine administration include intranasal, occular, buccal, genital tract (vaginal), rectal, intratracheal, skin, and gastrointestinal tract administration.

Preferably, vaccines of the invention are administered by the intranasal route. Without limitation to theory or particular mode(s) of action, intranasal administration of vaccines of the invention is believed to be advantageous for enhancing immunity against influenza virus infection as the virus enters the host through mucosal surfaces of the upper and/or lower respiratory tracts. In addition, mucosal vaccination (e.g. intranasal vaccination) may induce mucosal immunity not only in the respiratory tracts but also in distant mucosal sites including the genital mucosa.

Intranasal vaccines of the invention can be formulated, for example, in liquid form as nose drops, spray, or suitable for inhalation, as powder, as cream, or as emulsion. Nebulised or aerosolised intranasal vaccine compositions may also be utilised. Administration of vaccines of the invention to mucosa of the upper and/or lower respiratory tract via inhalation of mists, powders, or sprays, or by intranasal administration of nose drops, swabs, powders, sprays, mists, aerosols, and the like is preferred.

Vaccines of the invention may comprise an adjuvant such as, for example, those described in the subsection above entitled “Pharmaceutical compositions”. Any suitable adjuvant may be included in a vaccine composition of the present invention and the adjuvant may be included in any suitable form (e.g. a powder, a solution, a non-vesicular solution, or a suspension).

Non-limiting examples of adjuvants suitable for inclusion in vaccines of the invention and methods for their preparation are also described in Ohagan (Ed), (2000), “Vaccine Adjuvants: Preparation Methods and Research Protocols (Methods in Molecular Medicine)”, Humana Press Inc. Specific examples of such adjuvants include, but are not limited to, aluminum hydroxide; polypeptide adjuvants including interferons, interleukins, and other cytokines; AMPHIGEN, oil-in-water and water-in-oil emulsions; and saponins such as QuilA.

Preferably, the adjuvant is a mucosal adjuvant effective in enhancing mucosal immunity and/or systemic immunity to immunogenic components administered via the mucosal route. Mucosal adjuvants may be broadly classified as those that facilitate vaccine delivery (e.g. liposomes, cochleates, live-attenuated vectors, poly D,L-lactide-co-glycolide or PLGA, chitans, DNA vaccines, mucoadhesives) to enhance the induction of protective immunity induced by other immunogenic components of the vaccine, and those having an immunostimulatory role (e.g. innate immunity associated toxin-based, cytokine-based etc.). Without limitation to a particular mechanism, it is postulated that the advantageous effects of mucosal adjuvants partially derive from an ability to assist the passage of immunogenic components in the vaccine across the mucosal barrier. Upon traversing the mucosal barrier, the mucosal adjuvant may enhance immunity, for example, by complement activation, the induction of cytokines, stimulation of antibody production or antibody type switching, stimulating antigen presenting cells, and/or influencing MHC class I and/or class II expression.

In certain embodiments, vaccines of the invention for intranasal administration are provided in a freeze-dried powder form capable of re-constitution immediately prior to use. Powder vaccine formulations of vaccines of the invention provide a means of overcoming refrigerated storage and distribution requirements associated with liquid-based vaccine stability and delivery. Dry powder formulations offer the advantage of being more stable and also do not support microbial growth.

As demonstrated herein, freeze-dried formulations comprising gamma-inactivated influenza viruses of the invention induce levels of heterosubtypic immunity similar to that of non-freeze-dried formulations. Vaccines of the invention may be freeze-dried using any suitable technique known in the art. For example, liquid preparations of gamma-irradiated influenza viruses of the invention may be frozen in a dry ice-isopropanol slurry and lyophilized in a freeze Dryer (e.g. Virtis Model 10-324 Bench, Gardiner, N.Y.) for a suitable time period (e.g. 24 hours).

In one embodiment, a dry powder nasal formulation of a vaccine of the invention is produced by generating spray-freeze-drying (SFD) particles (see, for example, Costantino et al., (2002), “Protein spray freeze drying. 2. Effect of formulation variables on particle size and stability”, J Pharm Sci., 91:388-395; Costantino, et al., (2000), “Protein spray-freeze drying. Effect of atomization conditions on particle size and stability”, Pharm Res., 17:1374-1383; Maa et al., (1999), “Protein inhalation powders: spray drying vs spray freeze drying”, Pharm Res, 16:249-254; Carrasquillo et al., (2001); “Non-aqueous encapsulation of excipient-stabilized spray-freeze dried BSA into poly(lactide-co-glycolide) microspheres results in release of native protein”, J Control Release, 76:199-208; Carrasquillo et al., (2001), “Reduction of structural perturbations in bovine serum albumin by non-aqueous microencapsulation”, J Pharm Pharmacol., 53:115-120; and U.S. Pat. No. 6,569,458). For example, aqueous solutions containing gamma-irradiated influenza viruses of the invention and 10% solids (e.g. trehalose) may be passed through a sprayer with atomizing nitrogen gas and droplets collected in trays containing liquid nitrogen then lyophilized in a Manifold Freeze-Dryer. The freeze-dried formulation may be re-constituted immediately prior to use.

Preferred devices for intranasal administration of vaccines of the invention are nasal spray devices (e.g. devices available commercially from Pfeiffer GmBH, Valois and Becton Dickinson). Non-limiting examples of suitable devices are described, for example, in Bommer, (1999), “Advances in Nasal drug delivery Technology”, Pharmaceutical Technology Europe, p26-33. Intranasal devices may produce droplets in the range 1 to 500 μm. Preferably, only a small percentage of droplets (e.g. <5%) are below 10 μM to minimise the chance of inhalation. Intranasal devices may be capable of bi-dose delivery, that is, the delivery of two subdoses of a single vaccine dose, one sub-dose to each nostril.

A vaccine of the invention may be administered to a recipient in isolation or in combination with other additional therapeutic agent(s). In embodiments where the vaccine is administered with other therapeutic agent(s), the administration may be simultaneous or sequential (i.e. vaccine administration followed by administration of the agent(s) or vice versa).

### Dosages

In general, a composition of the invention is administered in a manner compatible with the route of administration and physical characteristics of the recipient (including health status) and in such a way that it is elicits the desired effect(s) (i.e. therapeutically effective, immunogenic and/or protective).

For example, the appropriate dosage of a composition of the invention may depend on a variety of factors including, but not limited to, a subject's physical characteristics (e.g. age, weight, sex), whether the compound is being used as single agent or adjuvant therapy, the type of MHC restriction of the patient, the likelihood of influenza infection, the progression (i.e. pathological state) of influenza infection, and other factors that may be recognized by one skilled in the art. Various general considerations that may be considered when determining an appropriate dosage of a composition of the invention are described, for example, in Gennaro et al. (Eds), (1990), “Remington's Pharmaceutical Sciences”, Mack Publishing Co., Easton, Pa., USA; and Gilman et al., (Eds), (1990), “Goodman And Gilman's: The Pharmacological Bases of Therapeutics”, Pergamon Press.

In general, compositions of the invention may be administered to a patient in an amount of from about 0.01 micrograms to about 5 milligrams of active component(s) (i.e. gamma-irradiated viruses of the invention). In some embodiments, the active component(s) are administered in a range of from about 0.1 micrograms to about 1 mg. In other embodiments, the active component(s) are administered in a range of from about 0.1 micrograms to about 500 micrograms. In other embodiments, the active component(s) are administered in a range of from about 0.1 micrograms to about 100 micrograms.

One skilled in the art would be able, by routine experimentation, to determine an effective, non-toxic amount of gamma-irradiated influenza virus of the invention to include in a composition of the invention for the desired therapeutic outcome.

Generally, an effective dosage is expected to be in the range of about 0.0001 mg to about 1000 mg of active component(s) (i.e. gamma-irradiated viruses of the invention) per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Alternatively, an effective dosage may be up to about 500 mg/m² of active component(s) (i.e. gamma-irradiated viruses of the invention). Generally, an effective dosage is expected to be in the range of about 0.01 to about 500 mg/m², preferably about 0.01 to about 350 mg/m², more preferably about 0.05 to about 300 mg/m², still more preferably about 0.05 to about 250 mg/m², even more preferably about 0.05 to about 250 mg/m², and still even more preferably about 0.05 to about 150 mg/m².

Typically, in therapeutic applications, the treatment would be for the duration of the disease state or condition. Further, it will be apparent to one of ordinary skill in the art that the optimal quantity and spacing of individual dosages will be determined by the nature and extent of the disease state or condition being treated, the form, route and site of administration, and the nature of the particular individual being treated. Also, such optimum conditions can be determined by conventional techniques.

In many instances, it may be desirable to have several or multiple administrations of a composition of the invention. For example, compositions of the invention may be administered 1, 2, 3, 4, 5, 6, 7, 8, 9, 10, or more times. The administrations may be from about one to about twelve week intervals, and in certain embodiments from about one to about four week intervals. Periodic re-administration may be desirable in the case of recurrent exposure to a particular pathogen or allergen targeted by a composition of the invention.

It will also be apparent to one of ordinary skill in the art that the optimal course of treatment can be ascertained using conventional course of treatment determination tests.

Where two or more therapeutic entities are administered to a subject “in conjunction”, they may be administered in a single composition at the same time, or in separate compositions at the same time or in separate compositions separated in time.

In certain embodiments, the methods described herein involve the administration of gamma-irradiated influenza viruses of the invention (or compositions/vaccines comprising gamma-irradiated influenza viruses of the invention) in multiple separate doses. Accordingly, the methods for the prevention (i.e. vaccination) and treatment of influenza virus infection described herein encompass the administration of multiple separated doses to a subject, for example, over a defined period of time. Accordingly, the methods disclosed herein include administering a priming dose of gamma-irradiated influenza viruses of the invention (or composition/vaccine comprising the same). The priming dose may be followed by a booster dose. The booster may be for the purpose of revaccination. In various embodiments, compositions or vaccines of the invention may be administered at least once, twice, three times or more.

### Routes of Administration

Administration of gamma-irradiated viruses of the invention (including those in the form of compositions and vaccines of the invention) may be performed by any suitable route, including, but not limited to, the parenteral (e.g. intravenous, intradermal, subcutaneous or intramuscular), mucosal (e.g. oral or intranasal) or topical routes.

In a preferred embodiment, administration in accordance with the methods of the invention is by the intranasal route. Intranasal administration is believed to provide significant advantages over other routes of administration. For example, intranasal administration induces secretory IgA production at mucosal epithelium eliciting cross protection more effectively than serum IgG.

Without limitation to particular modes of action, it is believed that gamma-irradiation inactivates the virus by introducing strand breaks in the viral genome without significantly affecting the antigenic structure of viral particles. Gamma-irradiation is thought to maintain the integrity of viral surface antigens in such a way that the virus maintains the ability to attach to cell receptors and enter host cells in a non-pathogenic state. Hence, gamma-irradiated influenza viruses are able to infect target cells of the respiratory tract in a much more efficient manner than other inactivated forms of the virus. This is reflected in the Examples of the present specification which show that formalin-inactivated virus administered intranasally does not induce heterotypic immunity. Hence, the combination of gamma-irradiated influenza virus and intranasal administration is thought to offer several advantages including, but not limited to, 1) facilitating the binding of inactivated virus to tissue specific receptors, 2) allowing the induction of tissue specific immune responses, 3) reducing the systemic exposure to whole virus antigen, and 4) limiting the side effects associated with whole virus vaccines.

In certain embodiments, gamma-irradiated influenza viruses of the invention are provided for intranasal administration in a freeze-dried powder form capable of re-constitution immediately prior to use (see section above entitled “Cross-protective influenza vaccines”). Powder vaccine formulations of vaccines and compositions of the invention provide a means of overcoming refrigerated storage and distribution requirements associated with liquid-based vaccine stability and delivery. Dry powder formulations offer the advantage of being more stable and also do not support microbial growth.

Intranasal administrations for use in accordance with the methods of the invention can be formulated, for example, in liquid form as nose drops, spray, or suitable for inhalation, as powder, as cream, or as emulsion.

Typically, the gamma-irradiated influenza viruses (or compositions/vaccines comprising the same) are administered to the nasopharyngeal area for absorption by nasal mucosa, preferably without being inhaled into the lungs. Suitable devices for intranasal administration in accordance with the methods of the invention are described above in the section entitled “Cross protective influenza vaccines”).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### EXAMPLES

The invention will now be described with reference to specific examples, which should not be construed as in any way limiting.

### Example 1

### Gamma-Irradiated A/PC Induces Stronger Cross-Protective Cytotoxic T-Lymphocyte Responses Than Other Gamma-Irradiated Influenza Virus Strains

### (i) Materials And methods

### Mice

Nine- to ten-week-old female BALB/c mice were used in this experiment.

### Viruses And Cells

P815 mastocytoma cells were grown and maintained in EMEM plus 5% FCS at 37° C. in a humidified atmosphere with 5% CO₂.

Stocks of influenza A viruses (A/PR8[H1N1] (5×10³ HAU/ml), A/PC[H3N2] (5×10³ HAU/ml), A/JAP[H2N2] (1×10³ HAU/ml), A/WSN/33 [H1N1] (3.2×10³ HAU/ml) and A/X31[H3N2] (1×10³ HAU/ml) were grown in embryonated hen eggs. Virus stocks were prepared from allantoic fluid and stored in aliquots at −70° C.

### Virus Inactivation

For gamma-ray inactivation, influenza viruses received a dose of 1×10⁶ rad (10 kGy) from a ⁶⁰Co source. The virus stocks were kept frozen on dry ice during gamma-irradiation. Loss of viral infectivity was confirmed by titration of inactivated virus preparations in embryonated eggs.

### Hemagglutination Assay

Live virus preparations were serially two-fold diluted in a 100 μl volume on a 96-well U-bottom microtiter plate. A 0.5% chicken erythrocytes suspension was added to all wells and plates incubated for 30 minutes on ice. The methods used were adapted from those described by Szretter and colleagues (Szretter et al., (2006); see Unit 15G.1 entitled “Influenza: Propagation, Quantification, and Storage”, in Coico et al., (Eds), “Current Protocols in Microbiology”, (2000-2010), John Wiley and Sons, Inc.).

### Vaccination

Groups of BALB/c mice were immunized intraperitoneally (i.p.) with 10³ HAU of inactivated virus.

In addition to being a suitable administration rate for inducing memory cytotoxic T cells in the spleen (the target organ of choice for cytotoxic T cell recovery), the intraperitoneal route was used to allow larger volumes of virus-containing allantoic fluid to be administered. This was necessary so that equal antigenic doses for all virus strains could be given. The virus concentrations of the allantoic fluids between the strains varied greatly and was lowest for A/X31 (1×10³ HAU/ml).

As the strain comparison needed to be standardised to a comparable antigenic load, HA rather than plaque forming units were used to set antigenic dose. The former is a measure of total viral particles in the inoculum, the later a measure of number of actually infectious particles in the pre-irradiated samples. Same PFU titres are mostly not equivalent in virus particle content.

### Generation of Secondary In Vitro Cytotoxic T-Lymphocytes.

Two weeks after immunisation splenocytes from two mice per group were pooled and aliquots of 2.5×10⁷ lymphocytes were cultured in 10 ml MEM 10% FCS and 2ME in the presence of 5×10⁶ stimulator cells for 5 days in a humidified 5% CO₂ atmosphere at 37° C. Stimulators were splenocytes from naïve BALB/c mice treated with either 10⁻³ M K^{d} peptide (TYQRTALVT) for 1 hour at 37° C. or infected with 10⁴ HAU of either A/PR8 or A/PC for 1 hour at 37° C. and thoroughly washed prior to addition to responders.

### Cytotoxic T Lymphocyte (Tc Cell) Assays

Cell cultures were harvested and lymphocytes resuspended in 2.5 ml of medium and titrated in 4, 3 fold dilution steps on 96 well tissue culture plates and used as effector cells in ⁵¹Cr release assays, as described by Mullbacher et al. (see Mullbacher et al., (1991), “Alloreactive cytotoxic T-lymphocytes recognize MHC class I antigen without peptide specificity”, J. Immunol., 147 (6):1765-72). P815 target T-lymphocytes (4×10⁶ cells) were mock infected or infected with 10⁴ HAU of live A/PC or A/PR8, or treated with 100 μl 10⁻³ M K^{d} peptide and incubated for 1 hour in the presence of 100 μCi m1⁻¹ of ⁵¹Cr. Targets were washed twice and incubated with effector cells in an 8 hour ⁵¹Cr release assay. The level of radioactivity in the supernatant was measured using a 96 well plate Top Counter (Perkin Elmer). Specific lysis was calculated as mean percent lysis of triplicate wells and values were calculated using the formula: (experimental release−spontaneous release)/(maximal release−spontaneous release)×100.

### (ii) Results

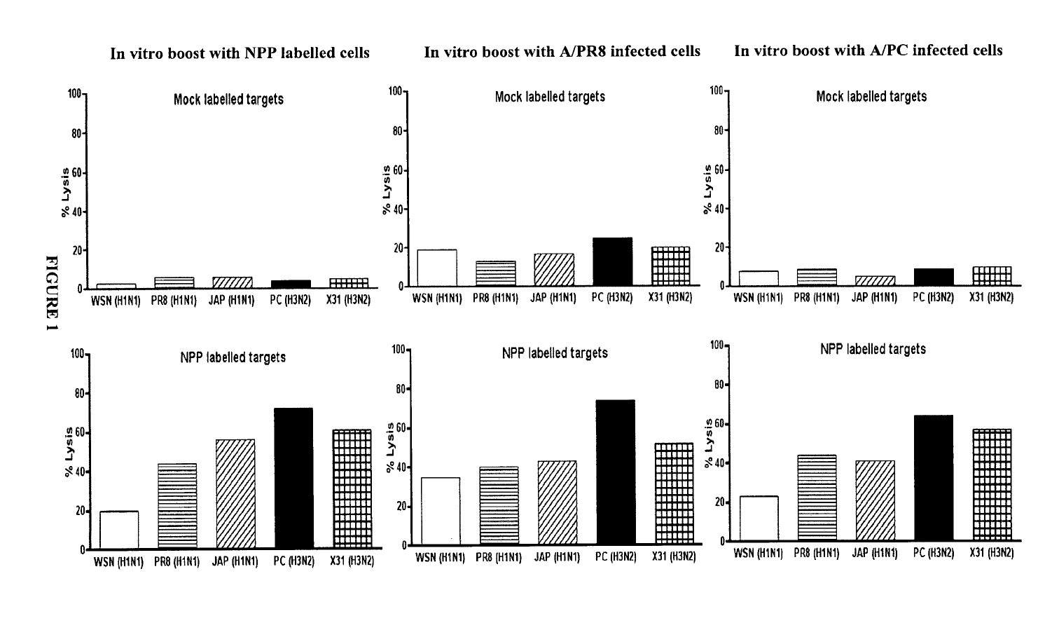

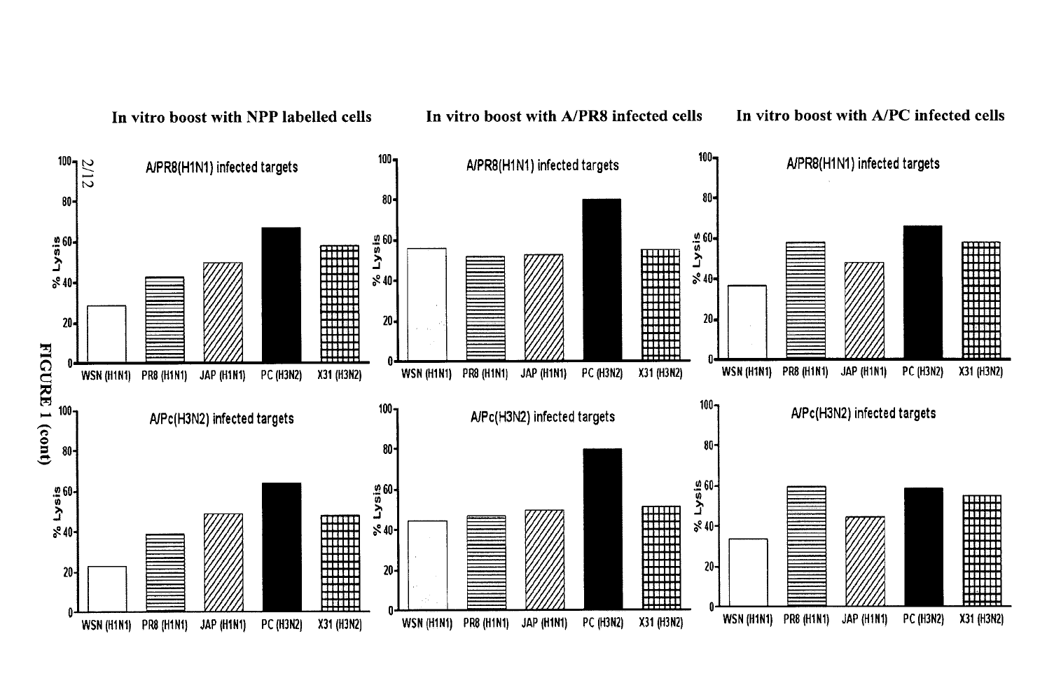

Groups of BALB/c mice were intraperitoneally vaccinated with 10³ HAU of five different strains of gamma-ray inactivated (10kGy) influenza A viruses. Two weeks later splenocytes from two mice per group were pooled and aliquots of 2.5×10⁷ live responder lymphocytes were co-cultured with 5×10⁶ stimulator cells for 5 days. Stimulator cells were either treated with 10⁻³ M peptide, the immunodominant, K^{d} restricted, CD8⁺ T-lymphocyte determinant derived from the nucleoprotein of A/influenza viruses (NPP) (TYQRTALVT), or infected with live A/PR8 or A/PC. Cultures were harvested and 1/25 of culture aliquots were titrated in four threefold dilution steps and tested for lytic activity against P815 target T-lymphocytes in a ⁵¹Cr release assay. Target T-lymphocytes were either mock infected, treated with NPP or infected with live A/PR8 or A/PC. Lysis values are presented in Table 1 and illustrated in FIG. 1. Lysis values were derived from regression analyses and resolved as percent specific lysis at 1/50 of culture aliquot.

It is important to note that lysis of peptide treated targets is solely due to the T-lymphocyte subset responsible for cross-protective lysis of different influenza virus strain-infected targets, which provides a measure of efficacy of cross-protection for gamma-irradiated influenza vaccine. Thus, based on lysis values of peptide treated targets obtained by effectors from cultures which were stimulated by peptide treated stimulators, the present data shows that mice vaccinated with A/PC have effectively generated the highest lytic potential in their spleen, the order of lytic potency being A/PC>A/X31>A/JAP>A/PR8>A/WSN (72; 61; 56; 44 and 20 respectively). Thus H3N2 gamma-inactivated viruses (A/PC and A/X31) (i.e. “gamma-A/PC” and “gamma-A/X31”) appear to be better vaccine candidates than H2N2 (A/JAP) or H1N1 (A/PR8 and A/WSN). This high percentage of lytic activity induced by gamma-irradiated A/PC is also evident in all other experimental settings regardless of secondary in vitro stimulation or influenza virus used for target infection.

TABLE 1
Lytic activity of splenocytes of mice
vaccinated with gamma-flu strains.

| | | | % specific lysis of P815 target | | | |
|---|---|---|---|---|---|---|
| in vivo | in vitro | live cells | T-lymphocytes | | | |
| gamma-flu | boost | recovered | MOCK | NPP K^{d 5} | A/PR8 | A/PC |
| A/WSN | NPP K^{d} | 8.5 × 10⁶ | 3 | 20 | 29 | 23 |
| A/WSN | A PR8 | 12.5 × 10⁶ | 19 | 35 | 56 | 45 |
| A/WSN | A/PC | 4.8 × 10⁶ | 8 | 23 | 37 | 34 |
| A/PR8 | NPP K^{d} | 10.3 × 10⁶ | 6 | 44 | 43 | 39 |
| A/PR8 | A PR8 | 13.0 × 10⁶ | 13 | 40 | 52 | 47 |
| A/PR8 | A/PC | 6.0 × 10⁶ | 9 | 44 | 58 | 60 |
| A/JAP | NPP K^{d} | 10.8 × 10⁶ | 6 | 56 | 50 | 49 |
| A/JAP | A PR8 | 12.0 × 10⁶ | 17 | 43 | 53 | 50 |
| A/JAP | A/Pc | 5.3 × 10⁶ | 5 | 41 | 48 | 45 |
| A/PC | NPP K^{d} | 9.5 × 10⁶ | 4 | 72 | 67 | 64 |
| A/PC | A PR8 | 14.5 × 10⁶ | 25 | 74 | 80 | 80 |
| A/PC | A/PC | 14.3 × 10⁶ | 9 | 64 | 66 | 59 |
| X31 | NPP K^{d} | 11.5 × 10⁶ | 5 | 61 | 58 | 48 |
| X31 | A/PR8 | 13.8 × 10⁶ | 20 | 52 | 55 | 52 |
| X31 | A/PC | 5.8 × 10⁶ | 10 | 57 | 58 | 55 |

### Example 2

### Intranasally Administered Gamma-Irradiated A/PC Induces Stronger Cross-Protective Immunity Than Gamma-Irradiated A/PC Administered Subcutaneously, Intraperitoneally, Or Intravenously

### (i) Materials and Methods

### Animals

10 week old BALB/c mice were utilised in this experiment.

### Viruses

Virus stocks of two A strains of influenza viruses (A/PR8 [H1N1] and A/PC [H3N2]), were grown in embryonated hen eggs and purified by temperature-dependent adsorption to chicken red blood cells, and virus titres estimated by standard plaque assays on Madin-Darby canine kidney (MDCK) cells and titres expressed as pfu/ml.

### Gamma-Irradiation of Influenza Strains

The purified stocks were exposed to 1×10⁶ rad (10 kGy) of gamma-rays. The residual viral infectivity in irradiated stocks was tested by using embryonated hen eggs. Virus stocks were sterile but retained full hemagglutinating activity after irradiation.

### Statistics

All statistical analyses were conducted using GraphPad InStat software. Fisher's exact and Chi Square tests were used to compare survival rates for significant differences.

### Intranasal Gamma Flu Vaccination Versus Other Routes of Administration

Different routes (intranasal (i.n.), intravenous (i.v.), intraperitoneal (i.p.) and subcutaneous (s.c.)) of inoculation were used to vaccinate BALB/c mice (10 mice/group) with 3.2×10⁶ pfu equivalent of gamma-A/PC. Three weeks post vaccination mice were challenged i.n. with a lethal dose of live A/PR8 (6×10² pfu) and monitored over a period of 21 days for mortality and clinical symptoms using a 30% body weight loss as the end point.

### (ii) Results

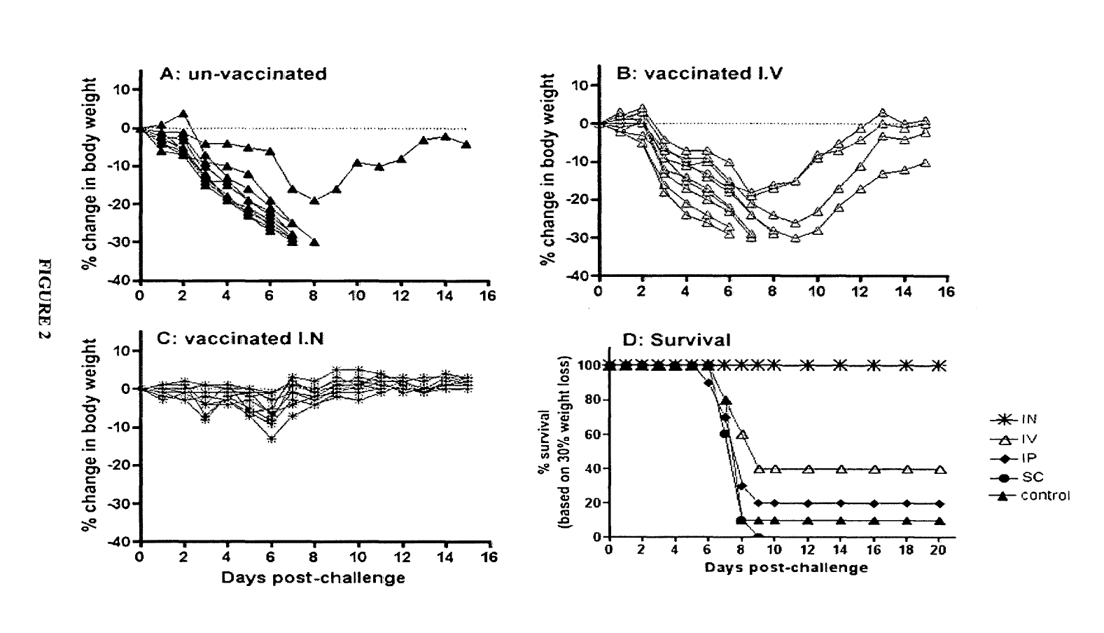

Intranasal gamma flu vaccination versus other routes of administration Different routes of inoculation (intranasal (i.n.), intravenous (i.v.), intraperitoneal (i.p.) and subcutaneous (s.c.)) were used to vaccinate BALB/c mice (10 mice/group) with 3.2×10⁶ plaque forming units (PFU) equivalent of gamma-A/PC. Three weeks post vaccination mice were challenged i.n. with a lethal dose of live A/PR8 (6×10² PFU) and monitored for mortality and clinical symptoms using a 30% body weight loss as the end point (FIG. 2).

All i.n. vaccinated animals fully recovered with little if any weight loss after challenge with heterotypic virus (FIG. 2C). In contrast, the majority of unvaccinated (FIG. 2A), i.v. vaccinated (FIG. 2B), and i.p. and s.c. vaccinated mice lost weight progressively to reach 30% body weight loss at days 7 and 8 post infection. The survival data (FIG. 2D) show that all i.n. gamma-A/PC vaccinated mice survived despite heterotypic challenge with unnaturally high challenge doses of A/PR8 (P<0.05 using Fisher's Exact test).

### Example 3

### A/PC Inactivated By Gamma Irradiation Induces Stronger Cross-Protective Immunity Than Ultraviolet- Or Formalin-Inactivated A/PC

### (i) Materials And Methods

### Mice

Nine- to ten-week-old female BALB/c mice were used in this experiment.

Viruses And cells

P815 mastocytoma, Madin-Darby canine kidney (MDCK) and baby hamster kidney (BHK) cells were grown and maintained in EMEM plus 5% FCS at 37° C. in a humidified atmosphere with 5% CO₂. The influenza type A viruses A/PR/8 [A/Puerto Rico/8/34 (H1N1)] and A/PC [A/Port Chalmers/1/73 (H3N2)] were grown in 10-day-old embryonated chicken eggs. Each egg was injected with 0.1 ml normal saline containing 1 hemagglutinin unit (HAU) of virus, incubated for 48 hours at 37° C., and maintained at 4° C. overnight. The allantoic fluids were then harvested, pooled and stored at −80° C. Titres were 10⁷ PFU/ml (A/PC) and 2×10⁸ PFU/ml (A/PR8) using plaque assays on MDCK cells. Viruses were purified using chicken red blood cells for vaccine preparation as described in (Sheffield, et al. (1954), “Purification of influenza virus by red-cell adsorption and elution”, British Journal of Experimental Pathology, 35:214-222). Briefly, infectious allantoic fluid was incubated with red blood cells for 45 minutes at 4° C. allowing the hemagglutinin to bind red blood cells, and then centrifuged to remove the allantoic fluid supernatant. The pellets were suspended in normal saline, incubated for 1 hour at 37° C. to release the red blood cells from the virus and then centrifuged to remove the red blood cells and collect the virus in the supernatant. Purified A/PC stock titre was 5×10⁸ PFU/ml.

### Virus Inactivation

For formalin inactivation, the viruses were incubated with 0.2% formalin at 4° C. for a week. The formalin was then removed by pressured dialysis using normal saline for 24 hours at 4° C. The dialysis method used was adapted from that described in Andrew et al. (Andrew et al., (2001); see Appendix 3H entitled “Dialysis and concentration of protein solutions” in Coligan et al., (Eds), “Current Protocols in Immunology”, (2000-2010), John Wiley and Sons, Inc).

For UV inactivation, the viruses were placed in 60-mm petri dishes with a fluid depth of 10 mm. The virus was exposed to 4000 ergs per cm² for 45 minutes at 4° C.

For gamma-ray inactivation, influenza viruses received a dose of 1×10⁶ rad (10 kGy) of gamma-rays from a ⁶⁰Co source. The virus stocks were kept frozen on dry ice during gamma-irradiation. Loss of viral infectivity was confirmed by titration of inactivated virus preparations in eggs.

The HAU titres of inactivated virus stock were determined to be 7.29×10⁴HAU/ml for gamma-A/PC, 2.43×10⁴ HAU/ml for formalin- and UV-A/PC.

### Freeze-Drying of Gamma-Inactivated Influenza

For freeze-drying, one vial containing 0.5 ml of gamma ray-inactivated A/PC was placed in a Manifold Freeze-Dryer (FTS SYSTEMS, Dura-Dry™ MP).

### Hemagglutination Assay

Live and inactivated virus preparations were serially diluted in a 100 μl volume in a 96-well U-bottom microtiter plate. 0.5% chicken red blood cell suspensions were added to all wells and plates were incubated for 30 minutes on ice. The methods used were adapted from those described by Szretter and colleagues (Szretter et al., 2006; see Unit 15G.1 entitled “Influenza: Propagation, Quantification, and Storage” in Coico et al., (Eds), “Current Protocols in Microbiology”, (2000-2010), John Wiley and Sons, Inc).

### Plaque Assay

Lung tissue samples were collected 3 and 6 days after intranasal challenge. After removal, whole lungs were homogenized in normal saline. Homogenates were centrifuged at 1500 rpm for 5 minutes. Supernatants were collected and were stored at −20° C. Serial dilutions of the samples were inoculated on MDCK cells cultured on 6-well tissue culture plates. After 1 hour adsorption, the cells were overlaid with EMEM medium containing 1.8% Bacto-Agar. After incubation for 2-3 days, cell monolayers were stained with 2.5% crystal violet solution and the plaques were enumerated.

### Lung-Histology

Lung tissue samples were fixed for a minimum of 24 hours in 10% neutral buffered formaldehyde. 10 μm sections were stained with Haemtoxilin-Eosin and evaluated by light microscopy.

### Cytotoxic T Lymphocyte (Tc Cell) Assay

Influenza-specific Tc cells were generated by intravenously injecting BALB/c mice with either live A/PC or inactivated 10⁸ PFU equivalent A/PC (gamma-irradiated, formalin inactivated, or UV inactivated). Spleens were harvested at 7 days post immunization and red blood cell-depleted cell suspensions were prepared for use as effector cells. Target T-lymphocytes were prepared by infecting P815 cells at a multiplicity of infection (m.o.i) of 1 for live A/PC and 10 for inactivated A/PC, followed by 1 hour incubation in medium containing 100˜200 μCi of ⁵¹Cr. After washing, target T-lymphocytes were mixed with effector cells at different ratios in an 8 hour chromium release assay. The level of radioactivity in the supernatant was measured in a gamma counter. Specific lysis is given as mean percent lysis of triplicate wells and values were calculated using the formula: (experimental cpm−spontaneous cpm)/(maximal release cpm−spontaneous cpm)×100.

### (ii) Results

### Effect of Virus Inactivation On Hemagglutination Activity

Hemagglutination activity after virus inactivation provides one indicator as to the denaturing effect of the sterilization treatment. Purified influenza stock was aliquoted into batches and treated with either formalin, UV or gamma irradiation. Following complete inactivation of infectivity verified by the absence of virus growth in embryonated eggs, the hemagglutination activity of live and inactivated viruses was compared (Table 2).

TABLE 2
hemagglutination activity of inactivated influenza virus A/PC preparations

| Strain | Method of inactivation | HAU/ml |
|---|---|---|
| A/PC | Original live purified stock | 2.2 × 10⁵ |
| | Gamma-ray inactivation | 7.3 × 10⁴ |
| | Formalin-inactivation | 2.4 × 10⁴ |
| | UV-inactivation | 2.4 × 10⁴ |

Hemagglutination activity was 3-fold reduced for gamma-irradiated viruses, whereas formalin and UV inactivation resulted in 9-fold reduced hemagglutination titres. These results provide evidence that, of these three virus sterilization methods tested, gamma-irradiation denatures viral protein structure the least.

### Gamma-Irradiated, But Not Formalin- Or UV-Inactivated, Virus Preparations Induce Heterosubtypic Immunity

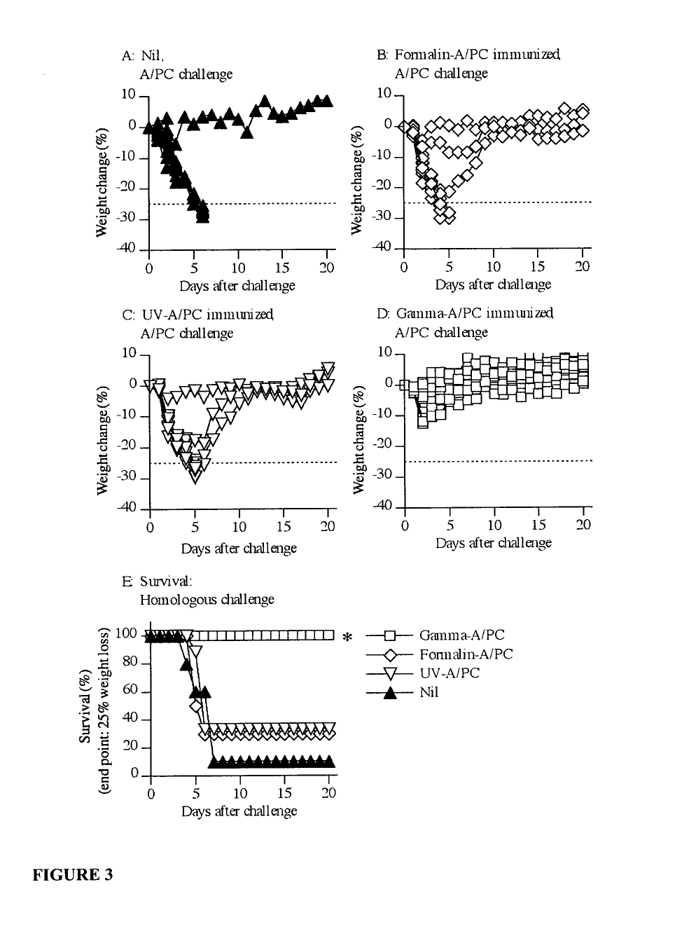

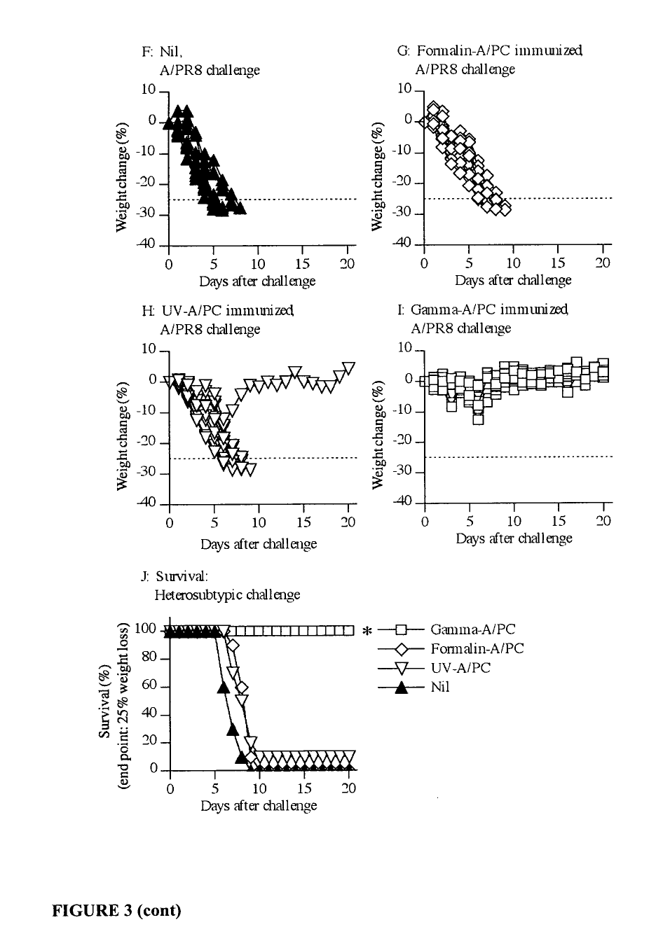

The protective efficacy of gamma-irradiated, formalin inactivated, or UV inactivated influenza virus preparations against homo- and heterosubtypic live virus challenges was compared. Groups of 9-10 BALB/c mice were mock treated or immunized intranasally either with formalin-, UV- or gamma-ray inactivated A/PC (3.2×10⁶ PFU equivalent) and at week 3 after the immunization, naïve and immunized mice (9-10 mice per group) were intranasally infected with A/PC (MLD-50; 3.2×10⁵ PFU) or A/PR8 (MLD-50; 7.0×10² PFU). Survival of infected mice was monitored daily for 20 days. As shown in FIGS. 3A, 3E, 3F, and 3J, intranasal infection of naïve mice with A/PC or A/PR8 caused a rapid weight loss with 90%-100% mortality (based on 25% weight loss as an end point). Mice immunized with either formalin inactivated A/PC (FIGS. 3B and 3E) or UV-inactivated A/PC (FIGS. 3C, 3E) also developed significant weight loss resulting in ˜70% mortality when challenged with live homologous virus. When similarly vaccinated mice were challenged with the heterosubtypic strain A/PR8, the animals lost substantial body weight with 90%˜100% mortality (FIGS. 3G, 3H, and 3J). In both cases (i.e. homologous and heterosubtypic challenge) the induced protection was considered inadequate to be useful for a vaccine (P-value>0.05, Fisher's exact test). In contrast, mice immunized with a single dose of gamma-inactivated A/PC were not only protected against homologous virus challenge, but also against heterosubtypic challenge, with mice losing only 5% of their body weight on average (FIGS. 3D, 3E, 3I, and 3J). Hence, gamma-irradiated influenza virus proved to be the most effective vaccine preparation to induce protective immunity against homo- and heterosubtypic influenza virus challenges (P<0.05).

### Minimal Influenza Infection-Induced Lung Inflammation After Vaccination With Gamma-Ray Inactivated A/PC

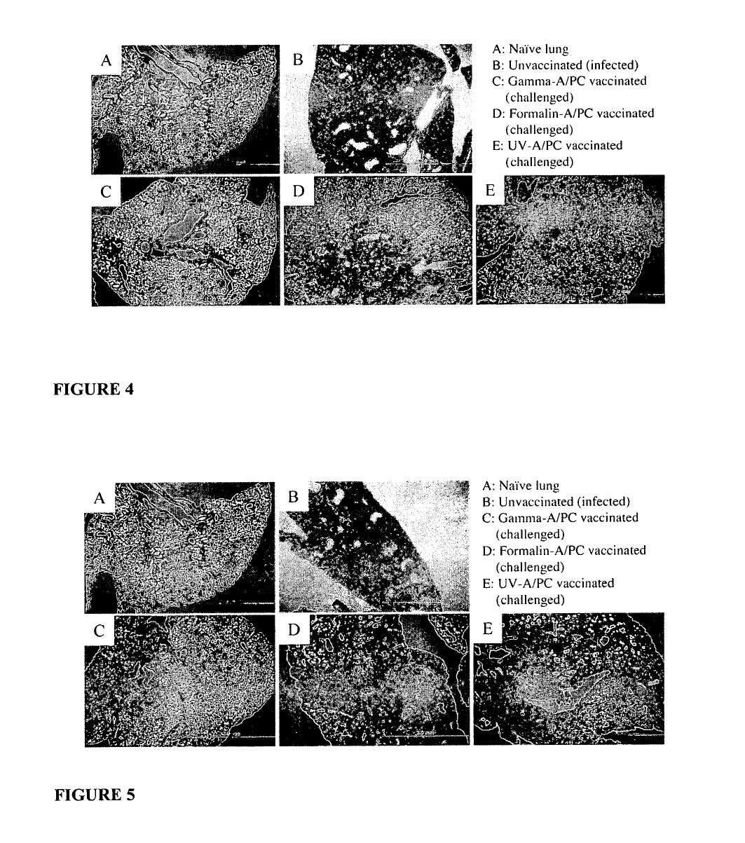

Three weeks following vaccination (3.2×10⁶ PFU equivalent) with gamma-irradiated, formalin- and UV-inactivated A/PC, mice were challenged with either live A/PC (homologous) or live A/PR8 (heterosubtypic). Lungs of surviving mice were harvested 21 days post-challenge and processed for histology. The lung samples displayed remarkable histological differences, corresponding to the type of immunization given. As shown in FIG. 4, limited inflammatory responses were seen when vaccinated mice were challenged with the homologous virus A/PC. Lung sections from all three vaccinated groups (gamma-irradiated, formalin- or UV-inactivated A/PC) were comparable in their appearance to that of naïve tissue (FIGS. 4A, 4C, 4D, and 4E). In contrast, lung tissues from unvaccinated, A/PC-challenged, mice showed extensive inflammatory responses (FIG. 4B). The heterosubtypic challenge resulted in clear differences among the various vaccinated groups.

The inflammatory responses in animals vaccinated with formalin- and UV-inactivated A/PC were strong and similar to those of unvaccinated animals following A/PR8 challenge 21 days post-vaccination (FIGS. 5B, 5D and 5E). In contrast, lung inflammation in gamma-irradiated A/PC vaccinated mice was limited following heterosubtypic challenge with A/PR8 (FIG. 5C). Although these lungs exhibited localised inflammation with weak lymphocyte infiltration, the overall condition was similar to that of naïve lungs (FIG. 5A).

### Gamma-Irradiated, But Not Formalin- Or UV-Inactivated Virus Retains Tc Cell Immunogenicity

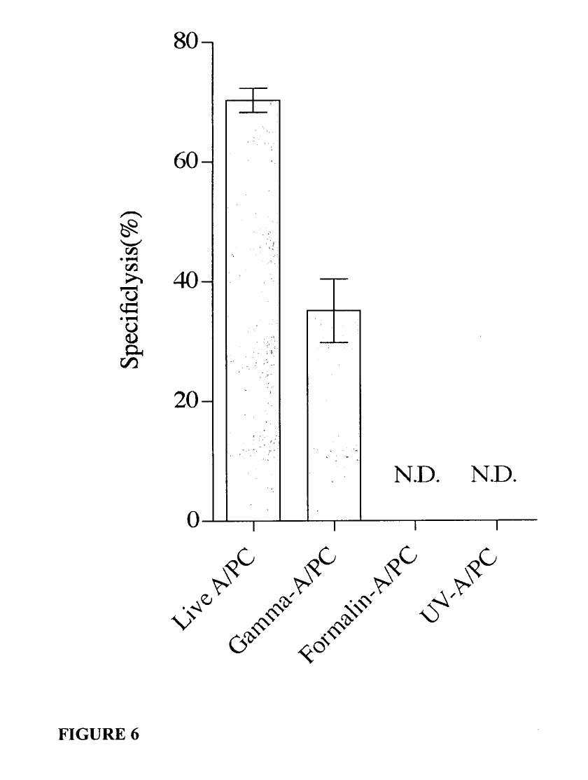

The ability to generate influenza-specific cytotoxic T (Tc) cell responses by challenge with live A/PC and inactivated A/PC (gamma-ray, formalin, and UV) was compared. BALB/c mice were intravenously immunized with live, gamma-irradiated, formalin-, or UV-inactivated A/PC. Splenocytes were harvested 7 days post immunization and were used as effector cells against A/PC infected P815 target T-lymphocytes. The peak of the Tc cell response following live virus infection was detected at day 7 post immunization (data not shown). On day 7 after intravenous immunization two mice from each group were assessed. Effector splenocytes harvested from mice immunized with live (10⁷ PFU) or gamma-ray inactivated A/PC (10⁸ PFU equivalent) lysed A/PC infected target T-lymphocytes, whereas effector cells from formalin- or UV-inactivated A/PC immunized mice did not (FIG. 6).

### Intranasal Immunization With Gamma-Ray Inactivated A/PC Confers Protection Against High Dose Heterosubtypic Challenges

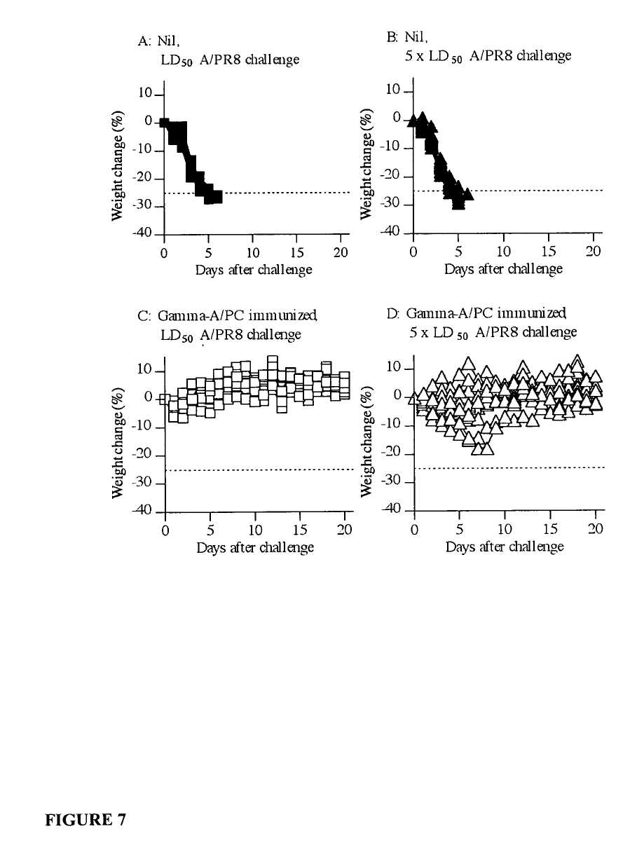

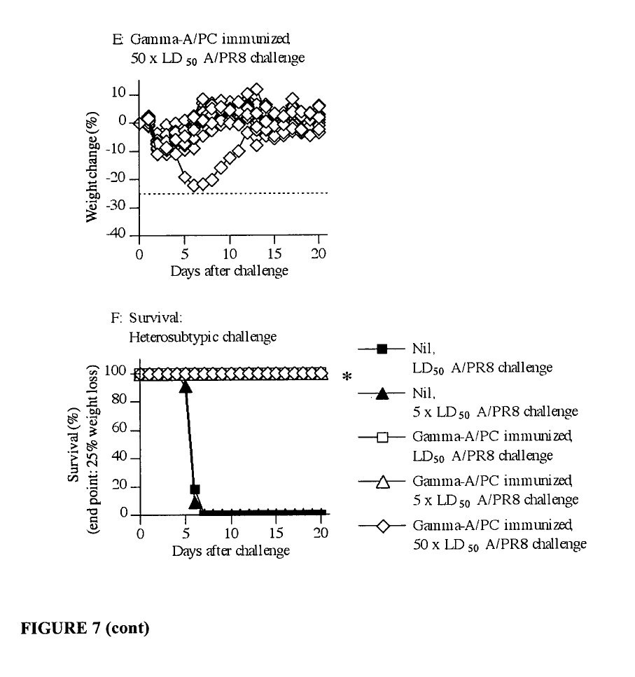

Given the excellent capacity of gamma-irradiated A/PC to protect mice from heterosubtypic challenge, the limit of protection was investigated by challenging with increased influenza virus doses (FIG. 7). Groups of 9-10 BALB/c mice were mock treated or immunized intranasally with gamma-ray inactivated A/PC (3.2×10⁶ PFU/ml equivalent) and at 3 weeks post immunization mice were intranasally challenged with either LD50 A/PR8, 5×LD50 A/PR8, or 50×LD50 A/PR8. Survival and weight loss of infected mice was monitored for 20 days. Immunized mice receiving heterosubtypic challenge of 1×LD50 all survived and there was little or no weight loss (FIGS. 7C and 7F). Immunized mice given a challenge dose of 5×LD50 initially lost weight during the first 7 days post-challenge (although not significantly) and all fully recovered (FIGS. 7D and 7F). The mice receiving 50×LD50 lost on average 8% of body weight but all mice fully recovered (FIGS. 7E and 7F). Naïve mice receiving 1×LD50 or 5×LD50 progressively lost weight and failed to survive the challenge (FIGS. 7A, 7B and F).

### Long-Lived Heterosubtypic Protection Conferred By Gamma-Ray Inactivated Preparations

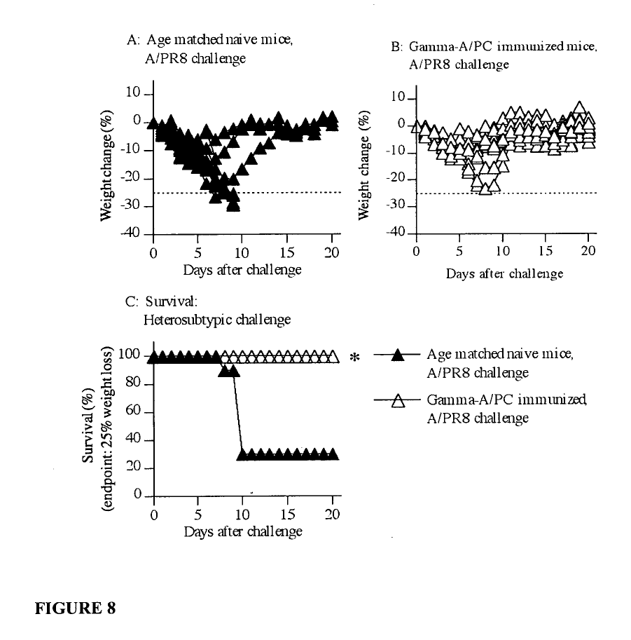

A critical requirement for an effective influenza vaccine is the induction of persistent heterosubtypic immunity. Groups of 9-10 BALB/c mice were either mock treated or immunized intranasally with gamma-ray inactivated A/PC (3.2×10⁶ PFU equivalent) and at 3 months post immunization mice were intranasally challenged with MLD-50 A/PR8 (7×10² PFU). Survival and weight loss of infected mice was monitored for 20 days. The vaccinated mice challenged with 1×LD50 A/PR8 lost on average only up to 10% body weight and fully recovered (FIGS. 8B and 8C). In contrast, the majority of challenged naïve mice lost substantial weight, reaching an end point of 25% total body weight loss at around 7 days post challenge (FIGS. 8A and 8C).

### Freeze-Drying Does Not Destroy the Immunogenicity of Gamma-Ray Inactivated A/PC

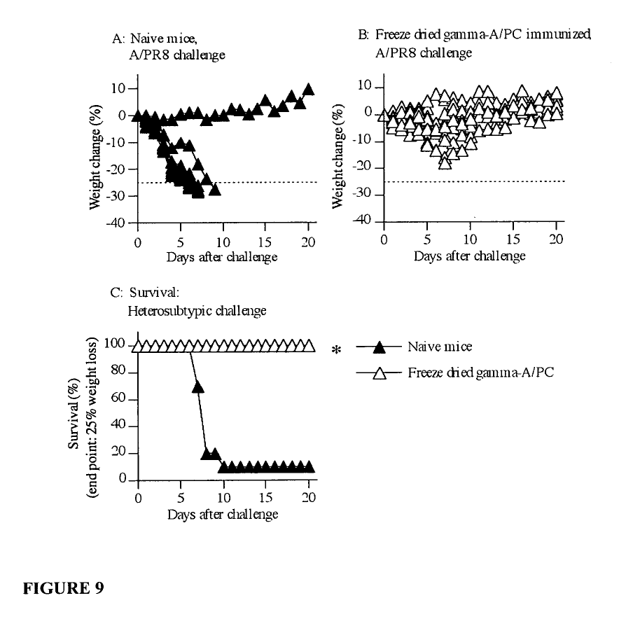

A known shortcoming of the current liquid based influenza vaccine is the requirement of refrigerated storage that imposes a problem for vaccine distribution, particularly in developing countries. In an attempt to overcome the stringent storage requirement of the current influenza vaccine, freeze-drying gamma-ray inactivated influenza virus was assessed as a means to curtail refrigeration requirements. Gamma-ray inactivated A/PC stock was freeze-dried and resuspended in distilled water immediately prior to intranasal administration (3.2×10⁶ PFU equivalent). Groups of 9-10 BALB/c mice were either mock treated or immunized with freeze-dried gamma-ray inactivated A/PC and challenged with heterosubtypic strain A/PR8 (7×10² PFU) at week 3 post-immunization. Survival and weight loss of mice was monitored daily for 20 days. The majority of mice lost less than 10% total body weight and only 2/10 mice lost over 10% total body weight showing mild symptoms. All vaccinated mice survived the heterosubtypic challenge with A/PR8 (7×10² PFU) as opposed to 10% survival in naïve mice (FIGS. 9A, 9B and 9C). These data suggest that the freeze-drying process does not markedly impact on the ability of gamma-ray inactivated A/PC to induce heterosubtypic immunity.

### Example 4

### Adoptive Transfer of T Lymphocytes From Mice Vaccinated With Gamma-A/PC Induces Cross-Protective Immunity

### (i) Materials And methods

Mice 10-week-old BALB/c female mice were used in this experiment.

### Cells And Viruses

P815 mastocytoma and Madin-Darby canine kidney (MDCK) cells were maintained in EMEM plus 5% FCS at 37° C. in a humidified atmosphere with 5% CO₂.

The influenza type A viruses, A/PR/8 [A/Puerto Rico/8/34 (H1N1)] and A/PC [A/Port Chalmers/1/73 (H3N2)] were grown in 10-day-old embryonated chicken eggs. Each egg was injected with 0.1 ml normal saline containing 1 hemagglutinin unit (HAU) of virus, incubated for 48 hours at 37° C., then held at 4° C. overnight. The amniotic/allantoic fluids were harvested, pooled and stored at −80° C. Titres were 10⁷ PFU/ml (A/PC) and 2×10⁸ PFU/ml (A/PR8) using plaque assays on MDCK cells. Viruses for vaccine preparation were purified using chicken red blood cells as described in (Sheffield, et al. (1954), “Purification of influenza virus by red-cell adsorption and elution”, British Journal of Experimental Pathology, 35:214-222). Briefly, infectious allantoic fluid was incubated with red blood cells for 45 minutes at 4° C. allowing the viral hemagglutinin to bind red blood cells, and then centrifuged to remove the allantoic fluid supernatant. The pellets were re-suspended in normal saline, incubated for 1 hour at 37° C. to release the red blood cells from the virus and then centrifuged to remove the red blood cells and collect the virus in the supernatant. Titres of purified stocks were 5×10⁸ PFU/ml for A/PC and 9×10⁸ PFU/ml for A/PR8.

### Virus Inactivation And Vaccination

Purified stocks of influenza viruses received a dose of 1×10⁶ rad (10 kGy) of gamma-rays from a ⁶⁰Co source. The virus stocks were kept frozen on dry ice during gamma-irradiation. Loss of viral infectivity was confirmed by titration of inactivated virus preparations in eggs.

Mice were immunized intranasally with inactivated virus preparations (3.2×10⁶ PFU equivalent) or live viruses at 70 PFU. For lethal challenge, at 3 weeks post-immunization, mice were infected intranasally with A/PR8 (7×10² PFU). Mice were weighed daily and monitored for morbidity until day 20 post-challenge.

### Adoptive Transfer of Immune Lymphocyte

10-week-old donor BALB/c mice were immunized intravenously (i.v.) with gamma-irradiated A/PC (1×10⁸ PFU equivalent). Splenocytes were collected at week 3 post immunization. Single-cell suspensions were prepared and red blood cells were lysed. The splenic lymphocytes were enriched into B and T-lymphocyte populations by passing the cells through nylon wool columns. 2 ml of cells (5×10⁷ cells/ml) were loaded onto columns and incubated for 2 hours at 37° C. The columns were washed with warm (37° C.) Hanks balanced salt solution+5% FCS and non-adherent T-lymphocytes in the first effluent were collected. Nylon wool-bound B-lymphocytes were then collected by washing the columns with cold (4° C.) Hanks balanced salt solution. Percentages of enriched T (82.8%, +7.94% B-lymphocyte) and B (84.2%, +8.3% T-lymphocyte) cell populations were determined by flow cytometric analysis. Small samples of enriched splenocytes were washed in PBS with 2% FCS. Fc receptors were blocked by incubation with anti-CD16/CD32 (Fey III/II receptor) Ab (BD Pharmingen) for 20 minutes at 4° C. Cells were washed and further incubated with a mixture of fluorescent-conjugated anti-CD3, anti-CD8, anti-CD19 (BD Pharmingen) antibodies. Dead cells were labelled with 7-aminoactinomycin D (Sigma-Aldrich). Stained cells were quantified using a FACS Calibur (Becton Dickinson). Enriched T or B-lymphocytes (1.1×10⁷ cells in a volume of 0.2 ml) were intravenously injected into recipient mice, which were then challenged intranasally with A/PR8 (7×10² PFU) 3 hours after the adoptive cell transfer. Mice were monitored for body weight loss and mortality until day 20 post-challenge.

### (ii) Results

### Adoptive Transfer of T Lymphocytes Provides Cross Protection

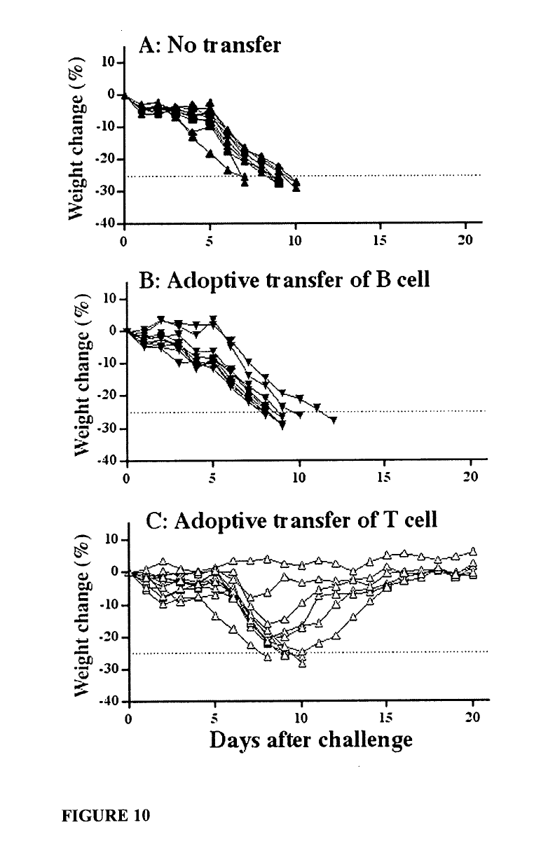

An alternative approach to assess the nature of the effector cells mediating cross-protection is adoptive transfer of different classes of influenza-specific lymphocytes. Mice were immunised with gamma-irradiated A/PC (1×10⁸ PFU equivalent) i.n. and three weeks later splenocytes were harvested and used as donor cells. Splenocytes were passed through nylon wool and enriched for T-lymphocytes (82.8% T-lymphocytes, 7.9% B-lymphocyte) or B-lymphocytes (84.2% B-lymphocytes, 8.3% T-lymphocytes) and transferred into naïve mice i.n. Three hours post-transfer, mice were challenged i.n. with A/PR8 (7×10² PFU/mouse). The majority of mice that received adoptively transferred T-lymphocytes were able to control the infection despite early weight loss (FIG. 10C). In contrast, no protection was observed in B-lymphocyte recipient mice, which developed disease symptoms similar to those of controls (unvaccinated with no lymphocyte transfer) following A/PR8 challenge (FIGS. 10A and 10B). These adoptive transfer studies support the notion that T-lymphocytes, but not B-lymphocytes, are critical for the cross-protective immunity induced by vaccination with gamma-irradiated influenza virus.

### Example 5

### Preparation of Influenza Virus For Gamma Irradiation By Tangential Filtration

Viruses used in current influenza vaccines are generally purified before attenuation using ultracentrifugation which has been associated with loss of viral-antigen and/or destruction of virion structure. The induction of cytotoxic T-lymphocyte responses by gamma-irradiated influenza vaccines will benefit from an alternative method of virus purification (differential/tangential filtration) prior to gamma-irradiation which preserves the integrity of virion structure.

It is envisaged that virus stocks will be clarified using centrifugation at low speed (˜3000 rpm) and used in size exclusion based centrifugation. Clarified stocks will be spun through a filtering device with pore size 50-80nm. In general, the size of influenza virus will be 80-120 nm. Thus, variable pore size (e.g. less than 80 nm) will be used to purify influenza virus at low centrifugation speeds (4000-10000 rpm) (variable speed can be used) at 4° C. for as long as needed to get liquid through the filter. The initial virus stock liquid flow path on the upstream side of the filter will be tangential or across the filter surface. Upon centrifugation, the majority of the liquid will pass through the filter (permeate), while a small portion will be retained in the central reservoir as the retentate (containing all the virus).

The retentate will be rediluted with PBS (normal saline, or any other media) that may contain sugar (dextran, sucrose) to maintain the osmotic pressure and consequently virus integrity. These diluted preparations may be filtered again, if needed. Concentrated virus from the final centrifugation step will be treated by gamma-irradiation as described in the Examples above. Free radical scavengers (e.g. ascorbate) can be added to purified virus stocks prior to irradiation to reduce possible damage to viral proteins while inactivating the viral genome using gamma-irradiation.

For example, the following protocol may be utilised for the purification of intact influenza virus to be used for gamma-irradiation:
1. influenza virus stock can be harvested from embryonated eggs, or in vitro tissue culture.
2. using filter devices with a cut off of 300 Kd, virus stock can be clarified by centrifuging at 300 g for 30 minutes at 4° C. causing both influenza viruses and proteins of the allentoic fluids (or tissue culture media) to pass through the filter.

3. using filter devices with a cut off of 100 Kd, clarified virus stock can be purified by centrifuging at 300 g for 30 minutes at 4° C. In this step influenza viruses do not pass through the filter (thereby concentrating the virus) on one side of the filter.

4. concentrated virus can be washed with normal saline (or any buffered media) to remove any remaining egg proteins (washing may be performed as many times as required). Washing can be performed by diluting the concentrated viruses with saline and centrifuging as described in step 3 above.

5. the final virus concentrate will contain intact virions.

In general, pore size cut off levels for filtering devices used in the above technique can be designed to match a virion size of 80-120 nm. All procedures may be conducted at 4° C. and no ultracentrifugation is required. Viral infectivity can be tested for original stock and final products. Prior knowledge of virus titres and volume can facilitate estimation of concentration levels. The purity of the final product can be determined using standard biochemical analyses.

1. A method for preventing or treating an influenza virus infection in a subject, the method comprising administering to the subject a therapeutically effective amount of a gamma-irradiated influenza virus, wherein the virus comprises an A/Port Chalmers/1/73 (A/PC) strain backbone of PB2, PB1, PA, NP, M1, M2 and NEP proteins.

2. A method for inducing or enhancing cross-protective immunity against multiple influenza virus subtypes in a subject, the method comprising administering to the subject a therapeutically effective amount of a gamma-irradiated influenza virus, wherein the virus comprises an A/Port Chalmers/1/73 (A/PC) strain backbone of PB2, PB1, PA, NP, M1, M2 and NEP proteins.

3. The method according to claim 1, wherein the influenza virus is strain A/PC.

4. The method according to claim 1, wherein the influenza virus comprises neuraminidase and hemagglutinin proteins from a strain other than A/PC.

5. The method according to claim 4, wherein the neuraminidase and hemagglutinin proteins are from an H5N1 subtype influenza A virus, an H1N1 subtype influenza A virus, HPAI A(H5N1) (bird flu), or pandemic H1N1/09 virus (swine flu).

6. (canceled)

7. (canceled)

8. (canceled)

9. The method according to claim 1, wherein the gamma-irradiated influenza virus is administered intranasally to the subject.

10. (canceled)

11. The method according to claim 1, wherein the influenza virus is prepared by gamma-irradiating a frozen viral preparation and/or is purified by tangential/cross-flow filtration prior to gamma irradiating the virus.

12. (canceled)

13. The method according to claim 1, wherein the gamma-irradiated influenza virus is prepared by exposure of the virus to a total dose of between about 6.5×10⁴ rad and about 2×10⁷ rad of gamma rays.

14. A method of producing a cross-protective influenza vaccine, the method comprising inactivating a preparation of influenza virus by gamma-irradiation, wherein the virus comprises an A/Port Chalmers/1/73 (A/PC) strain backbone of PB2, PB1, PA, NP, M1, M2 and NEP proteins.

15. The method according to claim 14, wherein the gamma-irradiated influenza virus is strain A/PC.

16. The method according to claim 14, wherein the influenza virus comprises neuraminidase and hemagglutinin proteins from a strain other than A/PC.

17. The method according to claim 16, wherein the neuraminidase and hemagglutinin proteins are from an H5N1 subtype influenza A virus, an H1N1 subtype influenza A virus, HPAI A(H5N1) (bird flu), or pandemic H1N1/09 virus (swine flu).

18. The method according to claim 14, wherein the vaccine is formulated for intranasal administration.

19. (canceled)

20. (canceled)

21. The method according to claim 14, wherein the preparation of influenza virus is gamma-irradiated while frozen and/or is purified by tangential/cross-flow filtration prior to inactivating the influenza virus by gamma irradiation.

22. The method according to claim 14, wherein the inactivating comprises exposing the influenza virus to a total dose of between about 6.5×10⁴ rad and about 2×10⁷ rad of gamma rays.

23.-25. (canceled)

26. The method according to claim 2, wherein the influenza virus is strain A/PC.

27. The method according to claim 2, wherein the influenza virus comprises neuraminidase and hemagglutinin proteins from a strain other than A/PC.

28. The method according to claim 27, wherein the neuraminidase and hemagglutinin proteins are from an H5N1 subtype influenza A virus, an H1N1 subtype influenza A virus, HPAI A(H5N1) (bird flu), or pandemic H1N1/09 virus (swine flu).

29. The method according to claim 2, wherein the gamma-irradiated influenza virus is administered intranasally to the subject

30. The method according to claim 2, wherein the gamma-irradiated influenza virus is prepared by exposure of the virus to a total dose of between about 6.5×10⁴ rad and about 2×10⁷ rad of gamma rays.
